SEQUENCE LISTING (27 sequences)

SEQ ID NO: 1 - DNA, 2244 bp, Pichia stipitis
  source 1..2244: mol_type = other DNA
atgtggaaatttctagaagctctactttacgacaacaccatcgaagaagaatactacaggaaaatccgtcaaaagtcctcctccaagtcggctgtaatcgtaggtcttgtagctgccgtgggaggctttttgtatgggtacgatacgggactcatcaacgacttgctagaaatgagatacgtctacgaaaactttccagaaaatctgcattcgttcacatcacatgaacgagcgttgattacggctgtgttatcgctcggaacattcataggagctctcatagcgcctcttatctccgacaactatggccggaagttttccatcattgtctcttccggtctcattttcaacgcaggcaacattttgcaaatcgcatcaacaaacgtagcattgctttgcgttggtagagcgatctcgggtgtatctgtaggcattctttcggccattgtacccttgtaccaagctgaagcttctcccaaatgggtcagaggttccgtcgttttcacatatcaatgggccattacttggggcttgttgatagcgagtgccgtctgtcaaggcactcgaaaaatgaccaattctggctcatatcggatccccgtgggcctccagtttctctgggctcttatcttgtacacggggatgcttttcttgcccgaaagtccccgttattatgttcaaaaagacgatcttcagaaagctctagatagtttgtcgaagttgcgaaagttgcccccagacgacgctgatttgatagaggagttggtggaaatcaaggctaactacgactacgagttgtcgtatggtaagaccaactatcttgattgcttccgtagtggaggaggaagacacaagcaggtgttgcgaatgttcactggaatcggtgctcaactctttcagcagtgttcaggcatcaacttcatcttctactatggtgtcaacttcttctccagcaccggcatccagaacttttacatcatgtccttcgtgacgtatttggtcaacactatcttcacaatccccggaataattctagtggatacgataggcaggcgacagttgctcctatggggtggcgtaggcatgtctaccgcgaacttcataattgcgattacgggagtcagtatctccagtaaggaaaccagttcgattctaagcgtctgtttttcgtgtgtgttcatagcgtttttcgccagttcgtggggtggatgtgtatgggcactcacttctgatatatacggtattagtatcagacagagagccatatccatcactacagccacgaactggttggtcaacttcatctttgcctacataacaccgtatctcatcgatacgggacaccatactgcagctataggaaacaaaatcttctttatctggggaggttgtaacgctgccggtgtcgttttcgtctacttcactgtctacgaaacaaagggattgaagttggaggaaattgattatatgtacgctcattgtgacaatgcgagaaagtccaccgagttcaagtcgaccaaaatcgattacactagattggacgagaactacaacgctgtaccctgggatcctccttatccatcaacaacgagctcatcgcctccttctaacatcaacgagaaggacctttcatcttctgatcccaaccaagacgtcaatgtacatagtgacaacaacgagtttgttccattgtacaacaacaaaaaacttccaaataatcctacaaacaccaacaaaaacgacatcaccatcattccctacaacaatatcattctgccgtcgttatcatcgaactccgagccctcttctgctgcttcgtcaattctcaacaacagattccaccacaactctgtctcgactacaaacaacgtctctgtatctacatctaaccctggccaatcttctggtcaaggtacagcttccaacgactacttgctgtatttggatagtttgaagtctgagtacggaagtccacctcactacaataacgacacactacaccagcagcacaccaaccaatcgaactccaagggttctgctacagacagaaacagcagcatcactgctagcaacattcaccatactcatagcaacatcaccagcaacattaataaccataatagtaataacattaataacagtataaccaacaattcgaccacgattattgccacgccatacttcaaccagcctccaccagactcttccgatgaagaagacgaagacgaggacgaagaagaatag

SEQ ID NO: 2 - DNA, 3141 bp, Urocitellus parryii
  source 1..3141: mol_type = other DNA
atggcttcatctgttggaaatgttgccgattctactgaacctaccaagagaatgttatcatttcaaggattagctgaattagcccatagggaataccaagccggtgactttgaagctgccgaaagacactgtatgcaattgtggagacaggagccagacaatacaggcgtgttattgttgttgtcatctattcatttccaatgtagaaggttggatagatctgctcactttagtactttggcaatcaaacagaatccattgttagctgaagcatattccaacttaggtaacgtctataaggaaagaggtcaattgcaggaagccatcgagcattaccgtcatgcattaagattgaagcccgatttcatcgatggttacattaacttagcagcagccttggttgcagcaggagatatggaaggtgcagtccaagcctatgtctccgcattacagtataaccctgacttgtactgtgttagatctgatttgggaaacttattgaaggcattgggaaggttggaagaggcaaaggcttgctacttgaaggccatagaaactcaaccaaacttcgcagtcgcatggtcaaacttgggttgcgtgttcaacgcacaaggtgaaatctggttagctatccaccatttcgagaaagcagttactttagatcccaatttcttggatgcctacatcaatttgggaaatgtgttaaaggaggcaaggatcttcgaccgtgctgtcgcagcatacttaagggctttgtctttaagtccaaaccacgcagttgtacatggcaacttggcttgtgtctactatgagcaaggattgattgacttagctattgatacctatcgtagggccattgaattacagccacactttcctgacgcttactgcaacttggctaatgccttgaaggagaagggatctgtggcagaggctgaggattgctacaacaccgcattgaggttatgtccaacacatgcagattccttaaacaatttggccaatatcaagcgtgaacagggtaacatcgaagaagccgtcagattgtatagaaaggccttagaggtctttcctgaatttgctgctgcccattccaacttagcaagtgtgttacagcagcaaggcaagttgcaagaagccttaatgcactataaggaagcaatcagaataagtcctacctttgccgatgcttattccaatatgggtaatactttgaaggaaatgcaggatgtccaaggtgccttacaatgttataccagggcaattcaaatcaatcctgctttcgcagatgctcattctaacttggcatccatccataaagattcaggcaacataccagaggccatcgcctcttatcgtacagccttgaagttgaagcctgatttcccagatgcctattgcaacttggcccattgtttgcaaattgtatgtgattggacagattacgatgaaagaatgaagaagttggtgagtatcgttgctgaccaattagagaagaacagattgccatcagttcaccctcatcactctatgttgtacccattgagtcatggtttcagaaaggcaattgctgaaaggcatggcaatttgtgtttggacaagatcaatgtcttacataagcctccatacgaacatcctaaggatttgaaattgtcagatggaaggttgcgtgtcggatatgtttcttctgatttcggtaatcatccaaccagtcacttaatgcagagtatccctggtatgcataatcccgacaaattcgaagtcttctgttatgccttaagtccagatgacggcaccaatttcagagtgaaagttatggctgaagctaatcatttcattgatttgagtcaaattccttgtaacggtaaagccgcagataggatacaccaagacggcattcatatcttggtcaatatgaacggatacacaaagggtgctagaaacgagttgtttgctttaagaccagcacctatacaagcaatgtggttaggctatccaggcacttcaggtgctttgttcatggactacatcataaccgaccaagaaacttctcccgcagaggttgctgaacaatacagtgagaagttggcttacatgccacatactttcttcattggtgatcatgccaatatgttcccacatttgaagaagaaagcagtcatagatttcaaatctaatggacatatctatgataacagaatagtgttaaacggtattgatttgaaagcattcttggattccttacctgatgttaagattgttaagatgaagtgtccagatggtggcgacaacgctgattcttctaataccgctttgaatatgcctgtgatcccaatgaatactatcgccgaggcagttatcgaaatgatcaatagaggccaaattcaaatcacaatcaatggattctctatttctaacggtttagcaacaacacagataaacaacaaagctgctaccggagaagaggtgcctaggaccattatcgttactacaagatcccagtatggtttacctgaggacgctatcgtgtactgcaacttcaatcagttatacaagatcgatccttcaaccttacagatgtgggctaacatcttgaagagggttcccaattcagtattgtggttgttaagatttcccgcagtcggtgagcctaacattcaacagtacgctcagaatatgggcttacctcagaatagaatcatattctctcctgtagctccaaaggaagaacatgtcagacgtggtcaattggccgatgtgtgtttggacactcccttgtgcaacggacacaccactggtatggatgtcttgtgggcaggcactcctatggtaactatgcctggcgaaacattggctagtagagttgcagctagtcaattgacatgcttaggttgcttagagttgattgcaaagaacagacaagaatacgaagatattgctgtcaagttaggtactgatttggagtacttgaagaaggttagaggtaaggtctggaaacaaagaattagtagtccattgttcaacaccaaacaatacacaatggaattagaaagattgtacttacaaatgtgggagcactatgccgcaggtaacaaacctgaccacatgatcaaaccagttgaagtaaccgaatccgcttaa

SEQ ID NO: 3 - DNA, 2751 bp, Rattus norvegicus
  source 1..2751: mol_type = other DNA
atggtacagaaggagagtcaagctgcattagaagaaagggaatcagaaaggaatgcaaacccagcatccgttagtggcgcatctttggaaccatctgccgctcctgctccaggtgaagataatccatcaggtgcaggcgctgccgcaggaactggagccgcaggaggagccaggagattcttatgtggtgtcgtagaaggcttctacggcagaccctgggttatggaacagagaaaggagttgttcagaagattgcagaagtgggagttgaatacttacttgtatgctcctaaggatgactacaagcatagaatgttctggcgtgaaatgtattctgtggaagaagctgaacagttgatgacattgatatcagctgcaagagaatacgaaattgagtttatctatgcaatctctccaggcttggacattaccttctccaacccaaaggaggtcagtaccttaaagcgtaagttggatcaagtgtctcaattcggatgtagaagtttcgccttattgttcgatgacatcgaccacaacatgtgcgctgctgacaaagaagtgttctcttctttcgcacatgcacaggtgtccatcacaaatgaaatctatcaatacttaggagaaccagaaacattcttattctgcccaacagagtattgtggcactttctgttatccatccgtcagtcaaagtccctatttgagaactgtgggtgagaagttattgccaggaatcgaagtattgtggactggacctaaagttgtgtctaaagaaattccagtggagtctattgaagaagtatcaaagatcataaagagagcacctgttatctgggataacattcacgctaacgattacgatcagaagagattgttcttgggtccttacaaaggtaggtctaccgagttgattccaaggttgaaaggcgtattgaccaatcctaactgtgaatttgaagcaaactatgtagccattcacacattggctacttggtataagtccaacatgaacggcgtacgtaaagatgttgttatgaccgattccgaagattccacagtatctatccagatcaaattggagaacgaaggtagtgacgaagatatagagactgatgtgttgtactctcctcagatggcattgaaattggccttgactgaatggttgcaggaatttggtgtgccacaccaatactcatcaagacaagtcgctcattccggagctaagacatctgttgtggatggcacacccttggtggcagcaccatctttgaatgcaacaacagttgttactacagtttatcaagaacccattatgtcacagggtgctgctttatccggtgagccctctgctttaactaaagaagaagagaagaaacaacctgatgaggaaccaatggatatggtggttgagaagcaagaagagtctgaacacaagtccgataaccagatcttaacagagattgtcgaagctaagatggcagaagaattgaaacctatggataccgataaagaatctattgccgaaagtaagagtccagaaatgagtatgcaagaagactgtatcaatgatattgcacccatgcagactgatgaacaagccaataaggaacagttcgttccaggcccaaatgagaagcctttgtatgcagcagaacctgtcactttggaagacttgcaattgttagcagatttgttctacttaccatacgagcacggtcctaagggtgcacaaatgttaagagaatttcaatggttaagagctaattcatctgtcgtgtcagtaaactgtaaaggtaaggatagtgagaagattgaagagtggagatcaagagcagccaagttcgaggaaatgtgtgctttagtcatgggcatgtttaccagattgtctaattgcgctaatagaactatcttgtatgatatgtattcatacgtttgggacataaagtccattatgtctatggtcaaatcatttgtacaatggttgggttgtagaagtcactcttctgcccagttcttgattggtgaccaagagccttgggcctttagaggcggattggcaggcgaatttcaacgtttgttacctattgacggtgctaatgatttgttctttcagccaccacctttaactcctacctctaaagtctatactattaggccatactttcccaaagacgaagccagtgtgtataagatctgtagagagatgtacgatgatggtgtaggtttgccatttcaatcccaaccagacttgataggtgacaaattggtaggaggtttgttgtctttatctttagactattgcttcgtgttagaggatgaggatggtatttgcggttatgccttaggaactgtagatgtaactcctttcataaagaaatgcaagatatcttggatacctttcatgcaagagaagtatactaaaccaaatggtgataaggaattgagtgaagctgagaagatcatgttatctttccatgaagaacaagaagtattgccagagacattcttggccaatttcccttctttgataaagatggatatacataagaaggtgactgatccttcagtagccaaatctatgatggcctgcttattgtcatccttaaaggccaatggatcaagaggtgctttctgtgaggtgaggccagatgacaagagaatcttagagttctatagtaagttaggttgtttcgagattgctaagatggaaggtttccctaaggacgtcgtgatcttgggaaggtctttgtaa

SEQ ID NO: 4 - DNA, 717 bp, synthetic construct
  misc_feature 1..717: note = Synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 5 - DNA, 16754 bp, synthetic construct
  misc_feature 1..16754: note = Synthetic construct
  source 1..16754: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccggtaccatgtggaaatttctagaagctctactttacgacaacaccatcgaagaagaatactacaggaaaatccgtcaaaagtcctcctccaagtcggctgtaatcgtaggtcttgtagctgccgtgggaggctttttgtatgggtacgatacgggactcatcaacgacttgctagaaatgagatacgtctacgaaaactttccagaaaatctgcattcgttcacatcacatgaacgagcgttgattacggctgtgttatcgctcggaacattcataggagctctcatagcgcctcttatctccgacaactatggccggaagttttccatcattgtctcttccggtctcattttcaacgcaggcaacattttgcaaatcgcatcaacaaacgtagcattgctttgcgttggtagagcgatctcgggtgtatctgtaggcattctttcggccattgtacccttgtaccaagctgaagcttctcccaaatgggtcagaggttccgtcgttttcacatatcaatgggccattacttggggcttgttgatagcgagtgccgtctgtcaaggcactcgaaaaatgaccaattctggctcatatcggatccccgtgggcctccagtttctctgggctcttatcttgtacacggggatgcttttcttgcccgaaagtccccgttattatgttcaaaaagacgatcttcagaaagctctagatagtttgtcgaagttgcgaaagttgcccccagacgacgctgatttgatagaggagttggtggaaatcaaggctaactacgactacgagttgtcgtatggtaagaccaactatcttgattgcttccgtagtggaggaggaagacacaagcaggtgttgcgaatgttcactggaatcggtgctcaactctttcagcagtgttcaggcatcaacttcatcttctactatggtgtcaacttcttctccagcaccggcatccagaacttttacatcatgtccttcgtgacgtatttggtcaacactatcttcacaatccccggaataattctagtggatacgataggcaggcgacagttgctcctatggggtggcgtaggcatgtctaccgcgaacttcataattgcgattacgggagtcagtatctccagtaaggaaaccagttcgattctaagcgtctgtttttcgtgtgtgttcatagcgtttttcgccagttcgtggggtggatgtgtatgggcactcacttctgatatatacggtattagtatcagacagagagccatatccatcactacagccacgaactggttggtcaacttcatctttgcctacataacaccgtatctcatcgatacgggacaccatactgcagctataggaaacaaaatcttctttatctggggaggttgtaacgctgccggtgtcgttttcgtctacttcactgtctacgaaacaaagggattgaagttggaggaaattgattatatgtacgctcattgtgacaatgcgagaaagtccaccgagttcaagtcgaccaaaatcgattacactagattggacgagaactacaacgctgtaccctgggatcctccttatccatcaacaacgagctcatcgcctccttctaacatcaacgagaaggacctttcatcttctgatcccaaccaagacgtcaatgtacatagtgacaacaacgagtttgttccattgtacaacaacaaaaaacttccaaataatcctacaaacaccaacaaaaacgacatcaccatcattccctacaacaatatcattctgccgtcgttatcatcgaactccgagccctcttctgctgcttcgtcaattctcaacaacagattccaccacaactctgtctcgactacaaacaacgtctctgtatctacatctaaccctggccaatcttctggtcaaggtacagcttccaacgactacttgctgtatttggatagtttgaagtctgagtacggaagtccacctcactacaataacgacacactacaccagcagcacaccaaccaatcgaactccaagggttctgctacagacagaaacagcagcatcactgctagcaacattcaccatactcatagcaacatcaccagcaacattaataaccataatagtaataacattaataacagtataaccaacaattcgaccacgattattgccacgccatacttcaaccagcctccaccagactcttccgatgaagaagacgaagacgaggacgaagaagaataggtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggtcgacgccaccatggcttcatctgttggaaatgttgccgattctactgaacctaccaagagaatgttatcatttcaaggattagctgaattagcccatagggaataccaagccggtgactttgaagctgccgaaagacactgtatgcaattgtggagacaggagccagacaatacaggcgtgttattgttgttgtcatctattcatttccaatgtagaaggttggatagatctgctcactttagtactttggcaatcaaacagaatccattgttagctgaagcatattccaacttaggtaacgtctataaggaaagaggtcaattgcaggaagccatcgagcattaccgtcatgcattaagattgaagcccgatttcatcgatggttacattaacttagcagcagccttggttgcagcaggagatatggaaggtgcagtccaagcctatgtctccgcattacagtataaccctgacttgtactgtgttagatctgatttgggaaacttattgaaggcattgggaaggttggaagaggcaaaggcttgctacttgaaggccatagaaactcaaccaaacttcgcagtcgcatggtcaaacttgggttgcgtgttcaacgcacaaggtgaaatctggttagctatccaccatttcgagaaagcagttactttagatcccaatttcttggatgcctacatcaatttgggaaatgtgttaaaggaggcaaggatcttcgaccgtgctgtcgcagcatacttaagggctttgtctttaagtccaaaccacgcagttgtacatggcaacttggcttgtgtctactatgagcaaggattgattgacttagctattgatacctatcgtagggccattgaattacagccacactttcctgacgcttactgcaacttggctaatgccttgaaggagaagggatctgtggcagaggctgaggattgctacaacaccgcattgaggttatgtccaacacatgcagattccttaaacaatttggccaatatcaagcgtgaacagggtaacatcgaagaagccgtcagattgtatagaaaggccttagaggtctttcctgaatttgctgctgcccattccaacttagcaagtgtgttacagcagcaaggcaagttgcaagaagccttaatgcactataaggaagcaatcagaataagtcctacctttgccgatgcttattccaatatgggtaatactttgaaggaaatgcaggatgtccaaggtgccttacaatgttataccagggcaattcaaatcaatcctgctttcgcagatgctcattctaacttggcatccatccataaagattcaggcaacataccagaggccatcgcctcttatcgtacagccttgaagttgaagcctgatttcccagatgcctattgcaacttggcccattgtttgcaaattgtatgtgattggacagattacgatgaaagaatgaagaagttggtgagtatcgttgctgaccaattagagaagaacagattgccatcagttcaccctcatcactctatgttgtacccattgagtcatggtttcagaaaggcaattgctgaaaggcatggcaatttgtgtttggacaagatcaatgtcttacataagcctccatacgaacatcctaaggatttgaaattgtcagatggaaggttgcgtgtcggatatgtttcttctgatttcggtaatcatccaaccagtcacttaatgcagagtatccctggtatgcataatcccgacaaattcgaagtcttctgttatgccttaagtccagatgacggcaccaatttcagagtgaaagttatggctgaagctaatcatttcattgatttgagtcaaattccttgtaacggtaaagccgcagataggatacaccaagacggcattcatatcttggtcaatatgaacggatacacaaagggtgctagaaacgagttgtttgctttaagaccagcacctatacaagcaatgtggttaggctatccaggcacttcaggtgctttgttcatggactacatcataaccgaccaagaaacttctcccgcagaggttgctgaacaatacagtgagaagttggcttacatgccacatactttcttcattggtgatcatgccaatatgttcccacatttgaagaagaaagcagtcatagatttcaaatctaatggacatatctatgataacagaatagtgttaaacggtattgatttgaaagcattcttggattccttacctgatgttaagattgttaagatgaagtgtccagatggtggcgacaacgctgattcttctaataccgctttgaatatgcctgtgatcccaatgaatactatcgccgaggcagttatcgaaatgatcaatagaggccaaattcaaatcacaatcaatggattctctatttctaacggtttagcaacaacacagataaacaacaaagctgctaccggagaagaggtgcctaggaccattatcgttactacaagatcccagtatggtttacctgaggacgctatcgtgtactgcaacttcaatcagttatacaagatcgatccttcaaccttacagatgtgggctaacatcttgaagagggttcccaattcagtattgtggttgttaagatttcccgcagtcggtgagcctaacattcaacagtacgctcagaatatgggcttacctcagaatagaatcatattctctcctgtagctccaaaggaagaacatgtcagacgtggtcaattggccgatgtgtgtttggacactcccttgtgcaacggacacaccactggtatggatgtcttgtgggcaggcactcctatggtaactatgcctggcgaaacattggctagtagagttgcagctagtcaattgacatgcttaggttgcttagagttgattgcaaagaacagacaagaatacgaagatattgctgtcaagttaggtactgatttggagtacttgaagaaggttagaggtaaggtctggaaacaaagaattagtagtccattgttcaacaccaaacaatacacaatggaattagaaagattgtacttacaaatgtgggagcactatgccgcaggtaacaaacctgaccacatgatcaaaccagttgaagtaaccgaatccgcttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatggtacagaaggagagtcaagctgcattagaagaaagggaatcagaaaggaatgcaaacccagcatccgttagtggcgcatctttggaaccatctgccgctcctgctccaggtgaagataatccatcaggtgcaggcgctgccgcaggaactggagccgcaggaggagccaggagattcttatgtggtgtcgtagaaggcttctacggcagaccctgggttatggaacagagaaaggagttgttcagaagattgcagaagtgggagttgaatacttacttgtatgctcctaaggatgactacaagcatagaatgttctggcgtgaaatgtattctgtggaagaagctgaacagttgatgacattgatatcagctgcaagagaatacgaaattgagtttatctatgcaatctctccaggcttggacattaccttctccaacccaaaggaggtcagtaccttaaagcgtaagttggatcaagtgtctcaattcggatgtagaagtttcgccttattgttcgatgacatcgaccacaacatgtgcgctgctgacaaagaagtgttctcttctttcgcacatgcacaggtgtccatcacaaatgaaatctatcaatacttaggagaaccagaaacattcttattctgcccaacagagtattgtggcactttctgttatccatccgtcagtcaaagtccctatttgagaactgtgggtgagaagttattgccaggaatcgaagtattgtggactggacctaaagttgtgtctaaagaaattccagtggagtctattgaagaagtatcaaagatcataaagagagcacctgttatctgggataacattcacgctaacgattacgatcagaagagattgttcttgggtccttacaaaggtaggtctaccgagttgattccaaggttgaaaggcgtattgaccaatcctaactgtgaatttgaagcaaactatgtagccattcacacattggctacttggtataagtccaacatgaacggcgtacgtaaagatgttgttatgaccgattccgaagattccacagtatctatccagatcaaattggagaacgaaggtagtgacgaagatatagagactgatgtgttgtactctcctcagatggcattgaaattggccttgactgaatggttgcaggaatttggtgtgccacaccaatactcatcaagacaagtcgctcattccggagctaagacatctgttgtggatggcacacccttggtggcagcaccatctttgaatgcaacaacagttgttactacagtttatcaagaacccattatgtcacagggtgctgctttatccggtgagccctctgctttaactaaagaagaagagaagaaacaacctgatgaggaaccaatggatatggtggttgagaagcaagaagagtctgaacacaagtccgataaccagatcttaacagagattgtcgaagctaagatggcagaagaattgaaacctatggataccgataaagaatctattgccgaaagtaagagtccagaaatgagtatgcaagaagactgtatcaatgatattgcacccatgcagactgatgaacaagccaataaggaacagttcgttccaggcccaaatgagaagcctttgtatgcagcagaacctgtcactttggaagacttgcaattgttagcagatttgttctacttaccatacgagcacggtcctaagggtgcacaaatgttaagagaatttcaatggttaagagctaattcatctgtcgtgtcagtaaactgtaaaggtaaggatagtgagaagattgaagagtggagatcaagagcagccaagttcgaggaaatgtgtgctttagtcatgggcatgtttaccagattgtctaattgcgctaatagaactatcttgtatgatatgtattcatacgtttgggacataaagtccattatgtctatggtcaaatcatttgtacaatggttgggttgtagaagtcactcttctgcccagttcttgattggtgaccaagagccttgggcctttagaggcggattggcaggcgaatttcaacgtttgttacctattgacggtgctaatgatttgttctttcagccaccacctttaactcctacctctaaagtctatactattaggccatactttcccaaagacgaagccagtgtgtataagatctgtagagagatgtacgatgatggtgtaggtttgccatttcaatcccaaccagacttgataggtgacaaattggtaggaggtttgttgtctttatctttagactattgcttcgtgttagaggatgaggatggtatttgcggttatgccttaggaactgtagatgtaactcctttcataaagaaatgcaagatatcttggatacctttcatgcaagagaagtatactaaaccaaatggtgataaggaattgagtgaagctgagaagatcatgttatctttccatgaagaacaagaagtattgccagagacattcttggccaatttcccttctttgataaagatggatatacataagaaggtgactgatccttcagtagccaaatctatgatggcctgcttattgtcatccttaaaggccaatggatcaagaggtgctttctgtgaggtgaggccagatgacaagagaatcttagagttctatagtaagttaggttgtttcgagattgctaagatggaaggtttccctaaggacgtcgtgatcttgggaaggtctttgtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 6 - DNA, 1155 bp, Saccharomyces cerevisiae
  source 1..1155: mol_type = other DNA
atgtccaagagcaaaactttcttatttacctctgaatccgtcggtgaaggtcacccagacaagatttgtgaccaagtttctgatgctattttggacgcttgtttagaacaagatccattctccaaggttgcctgtgaaacagctgccaaaactggtatgattatggttttcggtgaaattaccaccaaagctagacttgactaccaacaaatagtaagagataccatcaagaagattggttatgacgattctgccaagggtttcgactacaagacatgtaatgttttagtagctatcgaacaacaatctccagatatcgctcaaggtctgcactatgaaaagagcttagaagacttaggtgctggtgaccaaggtataatgtttggttacgctacagacgaaactccagaagggttaccattgaccattcttttggctcacaaattgaacatggctatggcagatgctagaagagatggttctctcccatggttgagaccagacacaaagactcaagtcactgtcgaatacgaagacgacaatggtagatgggttccaaagaggatagataccgttgttatttctgctcaacatgctgatgaaatttccaccgctgacttgagaactcaacttcaaaaagatattgttgaaaaggtcataccaaaggatatgttagacgaaaataccaaatatttcatccaaccatccggtagattcgtcatcggtggtcctcaaggtgacgctggtttgaccggtagaaagattattgtcgacgcttacggtggtgcctcatccgtcggtggtggtgccttctccggtaaggactattccaaggtcgatcgttccgctgcttacgctgctagatgggttgccaagtctctagttgccgctggtttgtgtaagagagtccaagtccaattttcatatgctattggtattgctgaaccattgtctttacatgtggacacctatggtacagctacaaaatcagatgacgaaatcattgaaattattaagaagaacttcgacttgagaccaggtgtgttagtaaaggaattagatttggctagaccaatttacttaccaaccgcttcttatggtcacttcactaatcaagagtactcatgggaaaaaccaaagaaattggaattttaa

SEQ ID NO: 7 - DNA, 1503 bp, Saccharomyces cerevisiae
  source 1..1503: mol_type = other DNA
atgtctagattagaaagattgacctcattaaacgttgttgctggttctgacttgagaagaacctccatcattggtaccatcggtccaaagaccaacaacccagaaaccttggttgctttgagaaaggctggtttgaacattgtccgtatgaacttctctcacggttcttacgaataccacaagtctgtcattgacaacgccagaaagtccgaagaattgtacccaggtagaccattggccattgctttggacaccaagggtccagaaatcagaactggtaccaccaccaacgatgttgactacccaatcccaccaaaccacgaaatgatcttcaccaccgatgacaagtacgctaaggcttgtgacgacaagatcatgtacgttgactacaagaacatcaccaaggtcatctccgctggtagaatcatctacgttgatgatggtgttttgtctttccaagttttggaagtcgttgacgacaagactttgaaggtcaaggctttgaacgccggtaagatctgttcccacaagggtgtcaacttaccaggtaccgatgtcgatttgccagctttgtctgaaaaggacaaggaagatttgagattcggtgtcaagaacggtgtccacatggtcttcgcttctttcatcagaaccgccaacgatgttttgaccatcagagaagtcttgggtgaacaaggtaaggacgtcaagatcattgtcaagattgaaaaccaacaaggtgttaacaacttcgacgaaatcttgaaggtcactgacggtgttatggttgccagaggtgacttgggtattgaaatcccagccccagaagtcttggctgtccaaaagaaattgattgctaagtctaacttggctggtaagccagttatctgtgctacccaaatgttggaatccatgacttacaacccaagaccaaccagagctgaagtttccgatgtcggtaacgctatcttggatggtgctgactgtgttatgttgtctggtgaaaccgccaagggtaactacccaatcaacgccgttaccactatggctgaaaccgctgtcattgctgaacaagctatcgcttacttgccaaactacgatgacatgagaaactgtactccaaagccaacctccaccaccgaaaccgtcgctgcctccgctgtcgctgctgttttcgaacaaaaggccaaggctatcattgtcttgtccacttccggtaccaccccaagattggtttccaagtacagaccaaactgtccaatcatcttggttaccagatgcccaagagctgctagattctctcacttgtacagaggtgtcttcccattcgttttcgaaaaggaacctgtctctgactggactgatgatgttgaagcccgtatcaacttcggtattgaaaaggctaaggaattcggtatcttgaagaagggtgacacttacgtttccatccaaggtttcaaggccggtgctggtcactccaacactttgcaagtctctaccgtttaa

SEQ ID NO: 8 - DNA, 765 bp, Saccharomyces cerevisiae
  source 1..765: mol_type = other DNA
atgagtatcgctgaattcgcttacaaggaaaaaccagaaactttggttttattcgatgttgatggtaccttgacaccagccagattaactgtttctgaagaagttagaaaaactttggccaagttgagaaacaagtgctgcattggttttgtcggtggttctgacttaagcaagcaattagaacagttaggcccaaacgttttagatgaatttgactattctttctctgaaaatggtttgaccgcctacagattaggtaaggaattagcttctcaatccttcatcaactggctcggtgaggaaaaatacaataaattggccgtcttcattttgagatatctatctgaaattgacttgccaaagagaagaggtactttcttggaatttagaaatggtatgatcaacgtttccccaattggtagaaatgcttctactgaggaaagaaacgaattcgaaagatacgataaggaacaccaaatcagagccaagttcgttgaagctttgaaaaaggaattcccagactacggtttgactttctccattggtggccaaatctctttcgacgttttccccgctggttgggataagacctactgtttgcaacacgttgaaaaagatggtttcaaggaaattcatttctttggtgacaagactatggtcggtggtaacgattacgaaatttttgtcgatgaaagaaccatcggacattcagtacaatcccctgatgacaccgtcaaaattttgactgaactattcaacttatag

SEQ ID NO: 9 - DNA, 1005 bp, Saccharomyces cerevisiae
  source 1..1005: mol_type = other DNA
atgttgtcaagagtagctaaacgtgcgttttcctctacagttgccaacccttataaagtgactgttttgggtgcaggcggtggtattggacaaccattgtctttgcttctaaagcttaaccataaagtcacggacttaagactgtacgacctaaagggcgcaaaaggtgttgccaccgatttgtctcatattccaacaaactccgtggtcaaggggtttactccagaagagccagacggattgaacaacgctttaaaggacacagacatggttttaattcctgctggtgtgcccagaaagcctggtatgacacgtgatgacttgttcgccatcaacgcaagcatcgttcgcgatttggcagcagcaaccgccgaatccgctcccaatgctgccattctggtcatttccaacccagtcaattctaccgttccaattgtcgcccaagtcttgaaaaacaagggtgtttacaacccaaagaaattgttcggtgtgactaccttggactctattagagccgccagattcatctcagaagtcgagaacaccgatccaactcaggaaagggttaacgtcatcggtggacattctggtattaccatcatcccattgatttcgcaaacaaaccataagttgatgtctgatgacaagagacacgaattgattcacagaatacagtttggtggtgacgaagtcgtcaaagcaaagaatggtgctggctctgctacgttgtcaatggcccatgctggtgctaaattcgctaacgctgttttgtccggtttcaaaggcgaaagagacgtcatcgagccttccttcgtggactctcccttgttcaaatccgaaggcatcgaattctttgcatctccggtcactttgggcccagatggtattgaaaagatccatccaataggtgagttatcttcagaagaagaagaaatgctacaaaaatgtaaagaaaccttgaagaagaatatcgaaaagggtgtcaactttgttgctagtaaatag

SEQ ID NO: 10 - DNA, 1458 bp, Saccharomyces cerevisiae
  source 1..1458: mol_type = other DNA
atggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaa

SEQ ID NO: 11 - DNA, 468 bp, Hordeum vulgare
  source 1..468: mol_type = other DNA
atgctcgtgtaccaggacaagctttccggcgatgaactcctgtcggattccttcccgtacagggagctggagaacggtgtgctctgggaagtcgatggccattgggtcgttcaaggagcagttgatgtggacattggtgccaacccctctgctgagggtggtggtgaggatgagggtgtcgatgaccaggccgtgaaggtggttgacattgttgacaccttccgtcttcaggagcaacctgcttttgacaagaagcagtttattgcttacatcaagcgctacatcaagaacctcactgccaagcttgaaggtgaggagctagatgctttcaagaagaacgttgagtctgccacgaagtatcttcttagcaagctcaaggaccttcagttctttgtgggcgagagcatgcatgatgatggcagcgtggtgtttaagcttgattgcctactacagggagggagctgctga

SEQ ID NO: 12 - DNA, 6438 bp, Saccharomyces cerevisiae
  source 1..6438: mol_type = other DNA
atgccagtgttgaaatcagacaatttcgatccattggaagaagcttacgaaggtgggacaattcaaaactataacgatgaacaccatcttcataaatcttgggcaaatgtgattccggacaaacgaggactttacgaccctgattatgaacatgacgcttgtggtgtcggtttcgtagcaaataagcatggtgaacagtctcacaagattgttactgacgctagatatcttttagtgaatatgacacatcgtggtgccgtctcatctgatgggaacggtgacggtgccggtattctgctaggtattcctcacgaatttatgaaaagagaattcaagttagatcttgatctagacatacctgagatgggcaaatacgccgtaggtaacgtcttcttcaagaagaacgaaaaaaataacaagaaaaatttaattaagtgtcagaagattttcgaggatttagctgcatccttcaacttatccgtattaggttggagaaacgtccccgtagattctactattttaggagacgttgcattatctcgtgaacctactattctacagccattattggttccattgtatgatgaaaaacaaccggagtttaatgaaactaaatttagaactcaattgtatcttttaaggaaggaggcctctcttcaaataggactggaaaactggttctatgtttgttccctaaacaataccaccattgtttacaagggtcaattgacgccagctcaagtgtataactactatcccgacttgactaatgcgcatttcaaatcccacatggcgttggtccattcaagattttccactaatactttcccctcttgggatagagctcaacctttacgttggctagctcataatggtgaaattaacaccttaagaggtaacaagaattggatgcgctccagagaaggtgtgatgaattcagcaactttcaaagatgagttagacaaactatacccaattatcgaagaaggtggttctgattcagctgcattggataacgttttagaactattgactattaatggcacattatctctacctgaagctgttatgatgatggttcctgaagcgtatcataaggatatggattctgacctaaaagcatggtacgactgggctgcatgtctgatggaaccttgggatggtccagctttgttaactttcactgatggacgttactgtggtgctatattggatagaaatggtttaagaccttgtcgttattacatcactagtgatgacagagttatctgtgcttcagaggtaggtgtcattcctatcgaaaattcattggttgttcaaaaaggtaaactgaagccaggtgatttattcctagtggatactcaattgggtgaaatggtcgatactaaaaagttaaaatctcaaatctcaaaaagacaagattttaagtcttggttatccaaagtcatcaagttagacgacttgttatcaaaaaccgctaatttggttcctaaagaatttatatcacaggattcattgtctttgaaagttcaaagtgacccacgtctattggccaatggttataccttcgaacaagtcacatttctgttaactccaatggctttaacaggtaaagaagctttaggttcgatgggtaacgatgcgccactggcttgtttaaatgaaaatcctgtcttactttatgattatttcagacaattgtttgctcaagtgaccaatcctccaattgacccaattcgtgaagcaaatgttatgtcgttagaatgttatgtcggacctcaaggcaaccttttggaaatgcattcatctcaatgtgatcgtttattattgaaatctcctattttgcattggaatgagttccaagctttgaaaaacattgaagctgcttacccatcatggtctgtagcagaaattgatatcacattcgacaagagtgagggtctattgggctataccgacacaattgataaaatcactaagttagcgagcgaagcaattgatgatggtaaaaagatcttaataattactgacaggaaaatgggtgccaaccgtgtttccatctcctctttgattgcaatttcatgtattcatcatcacctaatcagaaacaagcagcgttcccaagttgctttgattttggaaacaggtgaagccagagaaattcaccatttctgtgtcctactaggttatggttgtgatggtgtttatccatacttagccatggaaactttggtcagaatgaatagagaaggtctacttcgtaatgtcaacaatgacaatgatacacttgaggaagggcaaatactagaaaattacaagcacgctattgatgcaggtatcttgaaggttatgtctaaaatgggtatctccactctagcatcctacaaaggtgctcaaatttttgaagccctaggtttagataactctattgttgatttgtgtttcacaggtacttcttccagaattagaggtgtaactttcgagtatttggctcaagatgccttttctttacatgagcgtggttatccatccagacaaaccattagtaaatctgttaacttaccagaaagtggtgaataccactttagggatggtggttacaaacacgtcaacgaaccaaccgcaattgcttcgttacaagatactgtcagaaacaaaaatgatgtctcttggcaattatatgtaaagaaggaaatggaagcaattagagactgtacactaagaggactgttagaattagattttgaaaattctgtcagtatccctctagaacaagttgaaccatggactgaaattgccagaagatttgcgtcaggtgcaatgtcttatggttctatttctatggaagctcactctacattggctattgccatgaatcgtttaggggccaaatccaattgtggtgaaggtggtgaagacgcagaacgttctgctgttcaagaaaacggtgatactatgagatctgctatcaaacaagttgcttccgctagattcggtgtaacttcatactacttgtcagatgctgatgaaatccaaattaagattgctcagggtgctaagccgggtgaaggtggtgaactaccagcccacaaagtgtctaaggatatcgcaaaaaccaggcactccacccctaatgttgggttaatctctcctcctcctcatcacgatatttattccattgaagatttgaaacaactgatttatgatttgaaatgtgctaatccaagagcgggaatttctgtaaagttggtttccgaagttggtgttggtattgttgcctctggtgtagctaaggctaaagccgatcatatcttagtttctggtcatgatggtggtacaggtgctgcaagatggacgagtgtcaaatatgcgggtttgccatgggaattaggtctagctgaaactcaccagactttagtcttgaatgatttaagacgtaatgttgttgtccaaaccgatggtcaattgagaactgggtttgatattgctgttgcagttttattaggggcagaatcttttaccttggcaacagttccattaattgctatgggttgtgttatgttaagaagatgtcacttgaactcttgtgctgttggtattgccacacaagatccatatttgagaagtaagtttaagggtcagcccgaacatgttatcaacttcttctattacttgatccaagatttaagacaaatcatggccaagttaggattccgtaccattgacgaaatggtgggtcattctgaaaaattaaagaaaagggacgacgtaaatgccaaagccataaatatcgatttatctcctattttgaccccagcacatgttattcgtccaggtgttccaaccaagttcactaagaaacaagaccacaaactccacacccgtctagataataagttaatcgatgaggctgaagttactttggatcgtggcttaccagtgaatattgacgcctctataatcaatactgatcgtgcactcggttctactttatcttacagagtctcgaagaaatttggtgaagatggtttgccaaaggacaccgttgtcgttaacatagaaggttcagcgggtcaatcttttggtgctttcctagcttctggtatcacttttatcttgaatggtgatgctaatgattatgttggtaaaggtttatccggtggtattattgtcattaaaccaccaaaggattctaaattcaagagtgatgaaaatgtaattgttggtaacacttgtttctatggtgctacttctggtactgcattcatttcaggtagtgccggtgagcgtttcggtgtcagaaactctggtgccaccatcgttgttgagagaattaagggtaacaatgcctttgagtatatgactggtggtcgtgccattgtcttatcacaaatggaatccctaaacgccttctctggtgctactggtggtattgcatactgtttaacttccgattacgacgattttgttggaaagattaacaaagatactgttgagttagaatcattatgtgacccggtcgagattgcgtttgttaagaatttgatccaggagcattggaactacacacaatctgatctagcagccaggattctcggtaatttcaaccattatttgaaagatttcgttaaagtcattccaactgattataagaaagttttgttgaaggagaaagcagaagctgccaaggcaaaggctaaggcaacttcagaatacttaaagaagtttagatcgaaccaagaagttgatgacgaagtcaatactctattgattgctaatcaaaaagctaaagagcaagaaaaaaagaagagtattactatttcaaataaggccactttgaaggagcctaaggttgttgatttagaagatgcagttccagattccaaacagctagagaagaatagcgaaaggattgaaaaaacacgtggttttatgatccacaaacgtcgtcatgagacacacagagatccaagaaccagagttaatgactggaaagaatttactaaccctattaccaagaaggatgccaaatatcaaactgcgagatgtatggattgtggtacaccattctgtttatctgataccggttgtcccctatctaacattatccccaagtttaatgaattgttattcaagaaccaatggaagttggcactggacaaattgctagagacaaacaatttcccagaattcactggaagagtatgtccagcaccctgtgagggagcttgtacactaggtattattgaagacccagtcggcataaaatcggttgaaagaattatcattgacaatgctttcaaggaaggatggattaagccttgtccaccaagtacacgcactggctttacagtgggtgtcattggttctggtccagcaggtttagcgtgtgctgatatgttgaaccgtgccggacatacggtcactgtttatgaaagatccgaccgttgtggtgggttattgatgtatggtattccaaacatgaagttggataaggctatagtgcaacgtcgtattgatctattgagtgccgaaggtattgactttgttaccaacaccgaaattggtaaaaccataagcatggatgagctaaagaacaagcacaatgcagtagtgtatgctatcggttctaccattccacgtgacttacctattaagggtcgtgaattgaagaatattgattttgccatgcagttgttggaatctaacacaaaagctttattgaacaaagatctggaaatcattcgtgaaaagatccaaggtaagaaagtaattgttgtcggtggtggtgacacaggtaacgattgtttaggtacatctgtaagacacggtgcagcatcagttttgaatttcgaattgttgcctgagccaccagtggaacgtgccaaagacaatccatggcctcaatggccgcgtgtcatgagagtggactacggtcatgctgaagtgaaagagcattatggtagagaccctcgtgaatactgcatcttgtccaaggaatttatcggtaacgatgagggtgaagtcactgccatcagaactgtgcgcgtagaatggaagaagtcacaaagtggcgtatggcaaatggtagaaattcccaacagtgaagagatctttgaagccgatatcattttgttgtctatgggtttcgtgggtcctgaattgatcaatggcaacgataacgaagttaagaagacaagacgtggtacgattgccacactcgacgactcctcatactctattgatggaggaaagacttttgcatgtggtgactgtagaagagggcaatctttgattgtctgggccatccaagaaggtagaaaatgtgctgcctctgtcgataagttcctaatggacggcactacgtatctaccaagtaatggtggtatcgttcaacgtgattacaaactattgaaagaattagctagtcaagtctaa

SEQ ID NO: 13 - DNA, 2409 bp, Saccharomyces cerevisiae
  source 1..2409: mol_type = other DNA
atgctcaacattctcgttttaggaaacggtgcaagagaacacgttcttgtcaccaagctggctcagtcacccaccgtgggtaagatctatgtcgctccaggtaatggagggaccgcaaccatggatccttcgcgtgtgataaactgggatattacgccagatgtcgccaattttgctcgtttgcagtcgatggctgtggaacataagatcaacttggtcgttcctggtccagaattacctctagtcaacggcatcacctccgtgttccatagcgttggtattcccgtttttggaccttccgtcaaagccgctcagttggaagcttccaaggctttctccaagagatttatgtcaaaacacaatattccaaccgcgtcttatgatgtcttcactaatccagaagaagccatttcattcttgcaagctcatactgacaaagcttttgtcatcaaggccgacgggatcgctgctgggaaaggtgttattatcccatctagcatcgacgagtccgtccaagctatcaaggacataatggtcaccaagcaattcggtgaagaagcgggcaagcaggttgtgatagaacaattcttggaaggtgatgaaatctctctactcaccattgttgacgggtactctcacttcaatctccccgtcgcacaagatcacaagaggatctttgatggcgacaagggcttgaacaccggtgggatgggtgcctatgcccccgctcctgtggccacaccatctttgttgaagaccatagattcacagattgtgaagcctacgattgatgggatgagacgtgatggtatgccctttgttggtgtgctgttcaccgggatgattttggtgaaggattctaagacaaatcaacttgttcccgaagtgttagaatataatgtcagattcggtgacccagagacacaggctgttttgagtttacttgatgatcaaaccgatttggcgcaagtgtttttggctgctgctgaacatcgtttggattccgtaaacataggaatcgatgacacaagatctgccgttactgtcgtagtggctgcaggtggttatcctgaatcatacgccaaaggtgacaaaattaccttggataccgataaattacctccacatacacaaatcttccaagcaggtaccaaatacgattccgccaccgattctttattgaccaatggtggtagagttctttctgtgacctccactgctcaggacttgagaacagcagtagatacagtatatgaagccgtcaaatgcgtccatttccaaaattcttactacagaaaggacatcgcataccgtgcgttccaaaactcagaatcatcaaaagttgccatcacatacgcagactcaggtgtctctgttgataatggtaacaatctcgtacaaactatcaaagaaatggtcagatccacaagaaggccaggtgcagactctgatattggtggttttggtggtttattcgatttggctcaagcaggtttccgtcaaaacgaagataccttactagtaggtgctacagatggtgtcggtactaaattaatcattgcccaagagaccgggattcataatactgtcggtattgacctggtggccatgaatgttaacgatttggtggtacaaggtgctgagcctctattctttttggactactttgccactggtgctcttgacattcaagttgcctctgattttgtgtccggtgttgctaatggttgtattcaaagtggttgtgctcttgtgggtggtgaaacttcggaaatgcccggtatgtatccacccggccactacgatactaatggtaccgctgttggtgctgtattaagacaagatatcttgcccaagataaatgaaatggccgcaggagatgttcttctgggtctcgcctctagcggtgttcattctaatggtttctctttggttagaaaaattattcaacatgtagcattaccatgggacgctccatgtccatgggatgaatctaagacgttaggtgaaggtattcttgaaccaacaaaaatttacgtcaagcaattattgccatcaattagacaaagactactactaggtttagctcatataacaggtggtggtttagtagagaatatcccaagagctattccagaccacctacaggcccgcgttgatatgtcaacctgggaagtaccccgtgtcttcaaatggtttggtcaagcaggtaatgttccacacgatgacattttaagaaccttcaacatgggtgttggtatggttttgattgtcaagagagaaaacgtcaaggctgtttgtgattcattgactgaagaaggtgaaattatttgggagcttggttctttgcaagaaagaccaaaggatgctcccggttgtgtgattgaaaacggaactaagctttactaa

SEQ ID NO: 14 - DNA, 13047 bp, Mus musculus
  source 1..13047: mol_type = other DNA
cggttcactagaggagggaagaagtctacaaggacccatcaatttcctgcctgcctgcagcaaggacagacggcatgagcactgcgggaaaagtaaggaaaagatctcactgttggaaatgtattctcagtaatacgtggagagatgcagagtcctaccttagaaataacaggctctgatctgctgtagtgcagaaggtgtcctgggtgagcacagcttgacctgctactaaatatctttaacaagagggcatagaacctgggtttgtgaaagtctttattccctcacttcctctgcaggacagggaacttattccaagtcagtggtggctgctttgcaaacagaaagtttttaaaaaggtcttttactgaaagtctgtatttatcaataatgtgttattctttctggtcatattttgttatgaacatagaaaagttaaatctggcaagagtttcataggaggaatttgattcagggagcttagtactgttcctgtagccattaggtaacgtttctggtgagagatggactgatttgaaagcattagagtcattcacagtaagattatgttaccatgtaaattgtgatttgaagggcccattattgcacaactcacggatttctacaaaaatcctataaacagtctctctcctgtttaaaaaaaaatcatccagattttatggaaaattaatttgaataaaaatggaactgattgttagtattaagaatacacacatatggtactgagttttccacaaaaatcacactcatttgttcagcgtttacatggtaccgcaatgatggtgaacagccaatcagtataattaattatctatcgcttaattatataagcctatgtttctgttggcacagaggctattttaggagtgggtcaagagtccaacaactgacagtgagaactgggtgtccgacgtcgcagaggtttctgcacgcactgtgtgacaaaattcttcatttttatttcaattcacttcacactggttatgtttagggtggcatattacatttcaaagaaaaagcagttaggaaaaaaagttaaactgaaatttctagctgaaaaagtaaaaaaaaaaaaaaaagtaagttcatacagcaagaatgtaaacccaccacatccatcaaacagcaatcgagggaggggacagagaaagcagtctgaggtacagaggtcaataacagtgctgtgatgatagtcagtctatgcttatacttcctagagcagcaaaatcataatgtagccaggtatggtagaatatctctgaaatcttgggaataggaaggcccctagcttaaggccagcctgggctgcttattagaatttttgtctcagaagtcccacccctgggggttaactctgtgatagaggacttgcctagagtgcacaaagttctgggtactgtcctcggtgcctgaaaaataaataattataatttagaattaaatatatgcccaacaattgcacagaaacaacctttttggggacagtacaggtgacatccagaaagcaaaccaacaagcaagcagtcaaaacatggggcagcgtagcttgacagcacccgtgtcactttaacttggtatactacacttacttgtgttttcccgaccttgggctaagctgtcacacatttttttaaaggctttttaactctttgtaaatcagaactgtctgtctctatgtaacacttactgggtgacatagacggggacagatgatatatatatatatatatatatatatatatatatatgtgtgtgtgtgtgtgtatatatatatatatatatatatatatatatatatatacatacatacatatacatatacacacacatatatgtatgtatgtatatgtgtgcatgtatatgtatatgtatatgtgtatgtgtatgtgtatgtgtgtgtgtgtgtgtgtgtgtgtgtgtgtgtgtgtatgtgtatgtgtatatgtatatgtatatgtatatgtatatgtatatgtatatgtataatgtatatgtatatgtacatgaatgaaaggatgcaatcgcagtgatgtcattggagctgaagtaacccttcgctttccaggtgatcaaatgcaaagctgcggtgctatgggagcttcacaaacccttcaccatcgaggacatagaagtcgcaccccccaaggcccatgaagttcgaattaaggtgactgcccttttcaacttctgcaaagttaggttggaaaaactcgaaaagtactccatagccacatccagaaatccgttaatagcccctttcatgtgcttaattaatttgctgctatcaatatcaaatgtccaaagtttgaagtgactagaaagtatgtttaacctaatctcagaattttctttagatgcttatgtgtttatgaagtgctaaaacatcttgagtccaaaaccaactcatgttgggtaatgtcttggtgctaatgtttgatttctatttgcataaagcagcatgcattttcatatattccataaaacacattttcagagccacctactctacaggattgataattgagccatttaattgggaaaaggtaaacggaaacactggagaatcttggagataaatgaactctttggaatttggaagcagtaaggattgttaggaagcccctcagaagcacgactgcagacgaataggcttgtgaaacctccacaatcacaaacttggttaactggactttacctttaatccacagatggtggccactggtgtctgccgctcagacgatcacgtggttagtggaaccctggtcacacctcttcctgcagttttaggccatgagggagcaggcattgttgagagcgttggagaaggggtgacttgtgtgaaaccaggtacagaattcaccctcagggcttgtgtttcggttctgatctcaagagcatccctcccaagagaggaagttgaggaagtgggagatgaaaagccaggcaatagaaggcttcctctgccccttcctcccggcatgcctcagtcgtgctacatccaagcaagtcttttctctttcaacatctgtttatctcctacatgtcaggcatcaagccagaggctcctatatgaagatctggaagcacggtgcccgtaagggtgtgcagtttataatctgctaacttctactttattgtccctgagcgctgtggactttcaaaacccagacagaccttaaaaagtatccgaggcgggaaatgcaggtccaggttctgagatacacggcagctgttctgtgctgaacgcagagacgtgaaggcttgccctactcgcaggacgcccaccatagttttaaccacacactgggacaacatgatggcttcctcttttcctgtatgctccttctttatgaatatagataaaataaaatgtaagagggaatataagggaaaatcatagccaaaggccttcaaacccacagaccttcttatcattaattgtggctgtaggtggtaggtcacacagtatttattgcgttacacatggctccactttatatgtttttaaggtaaagggtgagcaaaacataaaatttccgtgataccatcccacattaattttatctatgaaatataatgaccaaacctttttttttggtccaataacataggtagcatacatgatttaaaaacaaaaattatgttcatcttcaaaaagatcgctttagtcaatagagtcagacttctcttctaaatgactaagctgtaagcaaaaaataggaaaagaactaatctggtttgagttaagcttgtattatttcttttattcagaaaagaacaaaacaacaacaacaaacctagcagcaaaatgacacctttactcttcaggttgcttcttctatggcagaaaaatcaataattcataaataattatctttaaaaaacaaaagtttcttgttatttcccagagaaagtatttaactcattaagaactgagatttccaaagcaatttgtggcttgtcatgtttttattatgcttcctcaaaaaataactaagattttttaagcctttatttcatagtaggagaagcacaatcaccttaaaaagccaaaactggaccacgaagaaatgataacgtggctcagggcataccatgctcttatagagcacccaactgggttactcacaaccacttgtaactccagctcctggggacccaacaccctttgctggcttccaagggcacacatacagagatggcacacacacacaggtactctcacatgcacacaaaaagaaaaaaaccaacaccaacaacaaagtcttaaacaataaaagggcaaaagctaactatcaagattcccactttaatttgatttcatgatcaagtttaatttgttttatttttactataaaatattgcctgtatagtgtttctttaatcttacttttattgaaactatcatatcagttctcgccaaaatctaacaggaagatagcaactcatttgcagagaaagtgtatatttcttcttagcagcaagcatatccaaggtttgattaggatatgtacaaagctcctaagagaaactgaggcacacaacaaagtttaactgctgacgggtggaaatggctggtttcggctcatttttctctcccgggaatcttcaggcgctttaaatgcgaatgactaattagactgacatagcctgggtgaggcctcatctcctggagacttttcaaagacacaatgtctctaataagcttcaagttgttgctttattgatttctggacagacatttaagtcattttgtttttcttcatccaggtgataaagtcattccactcttttcccctcagtgtggagaatgcaggatttgcaagcacccggaaagcaacttttgtagccgaagcgagtacgtttcttattgtcttcttgcacagttgggtgggcacactgctttgttctgtctcatgtcctttgtatgcctgtgtttcaccaaccagtctgctaatgcctcgggggactttgcgcgaaggcaccagcaggttctcctgcaagggaaagcagatccacaactttatcagcaccagcaccttctcccagtacaccgtggtagatgatatagcagtggccaaaatcgatggagcttcaccactggacaaagtctgcctcatcggctgtgggttctcaactggctatggctctgccgtcaaagtcgccaaggtaggatggacagtgggccatggaacaagctaagtgcatattattggtacctaaagggaagacatggctcctgggcgggcatcagagtatttttgtagaagtgaaataagcctctccagccccagtgagaattcactccacacacttgagagaagcaagggaaagctctataatagtgtgtgtctagttctagctcttgcccaaagagaaaccagcgtctaacagaaattatttgaagtttgtttaggtcttgggctgtaacattctacatatgccattgtctgcagatctctgttatgtcccagctgcaagactcactgagcaatgaaatacattttaaggagacagaaactggaaagcttgactatattggacaaatgggagattttataaagtggagatgccatcactgttatttctaaagcaagccacaatggggatatttgtactgcagagatagtgccacaagtgtgcgttagtttgtttttaagtagaccaacattgttacctagagccgtcctgccttccaggtagaattttctagataagaaaaccaaggcttttaatagcagagtagtactccattgtgtagatgtaccacattttctgtatctatccattcctctgttgaaagacatctgggttctttccagcttctggctattataaataaggctgttatgaacatagtggagcatgtgtccttgttatatgttggagcatcttctgtgtatatgcccaggagtatatgctgagtcctcatgtcaaatttctgaggaaccaacagactgatttccagagtggttgtaatcctaccaacaatggaggaatgtgcctctttctccacatcctcaccagcatctgctgaaattcttaggcaaatggatggaactagaaaatatcatcctgaatgaggtaacccaatcacaaaagaacacacatagtatgcattcactaataagtggggatattagccgcaaagcatggaatactcaagatacaattcacagatcacataaagctcaagaagaaggagacctaagtgtgggtccttcttagaaaggggaacaaaagactgttggaagccaatatagagatgaagcataaagcagaggctgaaggaaggtcatccagagaattcactccacacatgccccacttggggatccatcccaaatacagtcaccaaagccagacactattgtagatgccaagaagtgcatgctgacaggagcatgatacagctgtctcctgagaggccctgccagagccttacaaatacagatgcagatgctcaaagccaaccattggactgagagcagggccccaaatggaggatttagagaaagaacttaaggagccgaagggactaagacaccaaccaaagagtacacatggagggacccatggctcagccttatatgtaacagaggttggtcttgtcaggcatcaatgggaggagaggttcttggtcctgtgaaggctcgatagatgccccagtgtaggagaatgccagggtggtgaggtgggagtgggtgggtgggtgaaggaacaccctcatagaagcagggggagggggagtagtatagagggtttctagaagtgggggaaactggaaagggggataatatttgaaatgtaaataaaggaaatatccaattaaaaaatccttacatttaaaaaaaagaaagaaaaccaaggcttaatgatggaccaggatttaatggtaccatgacccagtgcagcattgtgagctgcctgacagaggcatgctctttgataatgggaagagcctagtggccagctgtgggctgtggttgtctctgtttgttaacatattcatgatgtcagaggcatgccttttcctatagtttctgacaagcctgcctagtgtgttggtcttccttaactcatgcaagcgacaacaccaggttagctaaactctactttgtgtgatataatatttcctaagcaagttcattagtagatccttcaagacatcaatttatcactctaggattctcctatgtcacttcatagatttatggttttgaattattaatagaaaaataaccccagatggaactggaaaaaaagaaaattcatcttttttttaaagatgaaaattatgctgattctgaattaagaaaaagtaaatatttataaatataataaatatttataataaatataatacatgtagaagatattataatctgtgtattctatatgactaccaaaatttaaagctgggaactcttgtagtagcctattctccagttgaaaagagatgtcactcatccatcatgatgggttccgctaggttctcaagtagccaaagcaaacggtttgtcttctcccagccttacctgattcatctgcagcatggccacccactgagtggatcccagaagactcaagagaacttaagcaaagtattgattacatttgagacctcatcactacatcttttcccttgcaaccaaaacacacaaacacacacacacacaaatacacatacgcatacaaacacacatatgcatacaaacacaaacagaaacatacacacaaacacactcataaccaacacacacacacacactagaagttttagatgtttagttttagacattttagatggcgattttaaaaataatttccttcccaaatggttgaaacaaatccagtagttatctttcattttatgaaaactaaaatccaggaagctaaagctaaaactattcatctaaattttcactagggaaataaagactagaattcctctcaacttctctgctgcatctgaaactagagtgcacactggtcatgactcccatcacagtaacaggccttgcatttttctgggccaggtggagagtggcgatgtgtcccagagcatttaagagcatttgatggatgaatgacaagatagacaccactagggggaagtgacagtcgggtatgggacacaggtggctgcaccaattaaatagacctaagccagtcacagtaatgaggagcctgccttttccaattctgaggctttagacctaaatgcaattcgtgcttgtgctgttgtttaactgtctcagctggaaggccgagtgtgcctatttgcaagccaagggtagttgtaatcatggattttaattaatcgtgactaaaatacagattagcttttgttgaagtatttgtcatttgcttcttctaaacattcaggcataaagtctcacagagattacattggtctcatgctatcttgtcttaaagtttcgtgtccactttcctattgctttaagccaggtatgacttctacatgcttctcagccgacttctgctcccagtaccgtagttgtgactactgaaagttagtgaaccaagaaaggagttcacgagcaaagggcagttgggttaccttttagtctcctggagtaaccttgatcacttgtttcatttattcaaattgcttatgtgcgtgtgtaagatgttattgtaaagctctcaaagatgtaagtcgtttttatttagaattcaaagatcatctggtacagctgatctcaacttgagttttccccattgaaataggaatgaggttccatttgcagtgattccaatttaattggtagaagctgctatcagaccttgagattaacgaaagaaatccccagatatgtctaatacatagcaaatcttgagtaccaatgatgtacactttgggcaaccataaccagaagtggatttggcaacaaatggaatgaggtagccgtgataaaggacacagcaaggcaatatgtgcagtggggagcaccccctaacagtcaccattcaatccacttttgtattttctggaaatacaggtgaccccaggctccacatgtgccgtgtttggcctcggaggtgtcggtctgtctgtcatcattggctgtaaagcagcaggagcagccaggatcattgctgtggacatcaacaaggacaagtttgccaaggccaaagagttgggtgcaactgagtgcatcaaccctcaagactacagcaaacccatccaggaagttctccaggagatgaccgacggaggggtggacttttcgtttgaagtcatcggccgccttgacaccatggtatgtactttggcacgccttgagatctgtccttccatctagaatgctctaggtagactaacagaaatctcatgcagaaagctatttttagagtggtcatcttccatctcctgtttcctgctcagactgcttaattcgctgttgagataaacctttcattttgtcagttctgcaaacttgtctcaagtgctaatcctcctttaatgcaacgaagctttcaatggggacactgtgaattaacttactgattttctgtaaaaaatcacttcatcgagcaggtttaaatacaaagtctggtcttaaatggatgaatatgattttctccctcattcttaatattttttaatatttagaattgaatattctgaaaagcatttttaagtatcatcatacggccaaaaggaataatgaacaatttggggcccaaggatcattttattaacacacaacacgtagagggaagtgactgatttatacatcaccatttagttctcgttgcagagacagcacagggctgggccttccaagcttccagatctaccggttgtagtaaataccttttaaaaaaagaattctggcatatctcctaaatttaagagatacattttataggattgcttatgatctccacaagacagtgcatataaaatactaactgtagagcccctgccatgtggtaagcactcagtaaaggtcagctgaggataatgataagaaatactacggtcaagcttagagcgattggattttggagtacaaatgacactacagtcaagtagttacaaatgtcatcctttgctaaagaactgttctccggttacatctcacctaaccaaagacctctagaactctactgagaaatgtccccagggagcaaggaagtcaccattatcaaggctctccacaaagtcttggcagagttggtgctggataataggtatggcgccctgagagtgatggtttggtttagttttttttccatccttgaactgtgacaagatgccatagactgtagaagattcaagtgcacagcacctcctctttccaaacccaggcttccagctctccccagagccactgctctcgtggctcactggtagatttctcaatctgctcatcttaagctgggctgtcaattgacttgaagatccaaaagtctgataaccacatgttctgaggatatctggtttctaaacatcatattgcaaaatcaaaggccactcatgtatctttaaggattccaaatgattttttactagagaatgtgttttaaacaaacaaacaaaaaaccaaacaaactaaagaaaagtacctattggaaggcaaaaacttcctggatgtctacagctatagaataatataaaactatcgcataaccacttaaaacatgcccattcttgatgtaagcacccgaggagggactcattaaatgagaatttgacaaatggttaaaagataagttttgatgatgtcatcatttatcttcttcaccattagatttctggctcagagtactagtttagaaattgtccttactgggcaaatgaaaagtgaactaagcagttcatagagagttatgcgatggggaaacaatattctcctttgagaaccagactttactttctcatgatgattcttgcacctttaaggaagaattaaacatagataagttaattcattcactctttcattaaaaaaagaaaaaaatcttcagtgtacttctttgtaatgcctgaaactgcatagtgaggagagaccacaaaagactctactttaactattactctttattccagacttctgccctgctgagctgccatgcagcatgtggtgtaagcgtcgtcgtaggagtgcctcccaatgcccagaacctctccatgaaccccatgttgctgctgctgggacgcacctggaagggagcaatatttggcggtatgtattcacagctcaagatcaatcctgcatctgtctgtatacgtcagggcgggcgtgtggatgtgtgtgtggaggacagagaacattcgattcattacctgggagccatttgccacgttctttgtgacagggtgtcacagtgaccaggctagccttgctggctggaatccactggttccctttatccagagctggaattacaagaactcccacaaccacatcagctgttggttggttggttggttggttggttggtcagttgttgttttgttattcaaactcaggtttgactgagccatctcctcagctttgaggatcaaatttttaatgtatttcagaatttcttcccttctgttacagagaaggtcagattttagaaggcaaaacaatttaaaaacatgaaaattactgttctctctaagcaagaactaatgcaggaaattgtaagacaagcttcattgtgtccccataaactagccagggcttcttctggctcctcctacttcctaatgtcattgtgagagccccaaaatgccttataggacacaggaaatccaaggcagtagtagtccatgcctataatcatgacctaagtctgaacagcggtgagaatggcaaagctcccttactttgagtacaaacatcttcaatatgatttctctaggaaagaactaataagccaccttcattagcgagagatcgcggtttagaggggtggcatacgataaaaatgttaatgaaatgcagaatgtttttgagcctcagtgtctgtgcagcctgcagaccactgttcttttatgaactttagtttccctgcaaagtctggttggcttgctccccccgccactcccccacacattccctccctccaaccccccaggctggatctcatggagatgttgctctccttccagggtttaagagtaaagattctgtccctaaacttgtggctgacttcatggctaagaagtttccgttggacccgttaattacccatgttttacctttcgagaaaataaatgaagcatttgacctgcttcgttctggaaagaggtaagctttgagattatttttatggcagaggaattggctaacagaaaatgaaggagaagtgggataagaccataaatgaagggggtgggggaaggggctggcttctgtcaccagtttggctctgcacacaggtatatgacataggacaattgcaaacctaagctttagtttccacagttcctaaaggtgacaatatgtgataatccaccttgtagtattgctgtttaggatgaggtttaaatgagataaccatgccttaaaagcttccataaagcacatggcaggtagcaaggaggaaatggctggtgttgcccattgttacacaagggatccagacttttagtgttgtttaagagtgctaaactagaaccggaatcaaaatggcagtcacatcaggatgttaagtgtgagtttagagctggagagaatgcaagagcgatcagaaaggtggattgctcgtttactgacttgaccttgggcatagcatggttgggaacccggatagattttctcagagactcctaacatgtcttctactcacttttatggtgagttatggaatgtgaaatcactatcttctgttctgtatttcagcatccgtaccgtcctgactttctgagatcatgtggatgccttcccacgcaccagtttctgaaccctaaaccagactgattcaagcaccagccacatcacagccttaatctttgctctttagagacacagccaataaagtacttgtgtaagctctcca

SEQ ID NO: 15 - DNA, 2024 bp, Arabidopsis thaliana
  source 1..2024: mol_type = other DNA
tctacttcttccaattaccagctgctatataaatccccttctctgtttctcttttcttacatcacaatcacacaaaactaacaaaagatcaaaagcaagttcttcactgttgataatgtctaccaccggacagattattcgatgcaaaggttttctttttattctgtctttttccaaatatttattgatcggttacatttctgttgaggtttttgttatgaatccacaatttctatgttgaattaacaaaacctgtgtcgtttttttgtggtggttgcagctgctgtggcatgggaagccggaaagccactggtgatcgaggaagtggaggttgctccaccgcagaaacacgaagttcgtatcaagattctcttcacttctctctgtcacaccgatgtttacttctgggaagctaaggtagagtaatcaatttattacactccaaattcataatcaagttctaatttttttagaattctaattttttatctaaaaaaattcaacctttttgattccacagggacaaacaccgttgtttccacgtatcttcggccatgaagctggagggtaatagaaacactaatcttctttgcttcgttttggatatttttaaggttttagagattcaaggtcgttttttttgttgttgtgtaggattgttgagagtgttggagaaggagtgactgatcttcagccaggagatcatgtgttgccgatctttaccggagaatgtggggagtgtcgtcattgccactcggaggaatcaaacatgtgtgatcttctcaggatcaacaccgagcgaggagggatgattcacgatggtgaatcaagattctccattaatggcaaaccaatttaccatttccttgggacttccacgttcagtgagtacacagtggttcactctggtcaggttgctaagatcaatccggatgctcctcttgacaaggtctgtattgtcagttgtggtttgtctactgggttaggagcaactttgaatgtggctaaacccaagaaaggtcaaagtgttgccatttttggtcttggtgctgttggtttaggcgctgcagaaggtgctagaatcgctggtgcttctaggatcatcggtgttgattttaactctaaaagattcgaccaaggtattcaaaaagatgatagtctgtttttgactatgttcttctataatctcccttcacttacattgaatttgatatgttattggcagctaaggaattcggtgtgaccgagtgtgtgaacccgaaagaccatgacaagccaattcaacaggtgatcgctgagatgacggatggtggggtggacaggagtgtggaatgcaccggaagcgttcaggccatgattcaagcatttgaatgtgtccacgatgtaatcctcccttcacatcattcggaccaaaacttttgtaactacattgtgggtatctgaacttatcacatatgatgttgtttcagggctggggtgttgcagtgctggtgggtgtgccaagcaaagacgatgccttcaagactcatccgatgaatttcttgaatgagaggactcttaagggtactttcttcgggaactacaaacccaaaactgacattcccggggttgtggaaaagtacatgaacaaggtaatgagaagctttgatatcttatgatgccaactttgaatatatatcaatgttctgatgatttttatgacataggagctggagcttgagaaattcatcactcacacagtgccattctcggaaatcaacaaggcctttgattacatgctgaagggagagagtattcgttgcatcatcaccatgggtgcttgaagccattctctcgcagatgatgttcactttgtgttttacttcctttatgcattcacagcaataaaagaaagaaatctccatcgcttttggttttcttctctgtcttaagttagtcgttttcgtgtctaatctattacttatcattgtaatagactcttcttctattgagatttgaatataaactaaaacacattccatttt

SEQ ID NO: 16 - DNA, 1445 bp, Saccharomyces cerevisiae
  source 1..1445: mol_type = other DNA
aagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaact

SEQ ID NO: 17 - DNA, 2655 bp, Saccharomyces cerevisiae
  source 1..2655: mol_type = other DNA
atggatcctaatagtaacagttctagcgaaacattacgccaagagaaacagggtttcctagacaaagctcttcagagggtgaagggcatagcactgcgacgaaacaatagtaacaaagatcatacaacagatgatacgacaggtagcatacgaacccctacgagcttgcagcggcaaaattctgacaggcaatctaatatgacatccgtgtttacggatgacatttctaccatagacgacaactcaattttattttcagagcctcctcagaaacaatctatgatgatgtctatatgcgtaggtgtttttgttgcagttggcggatttttatttggttatgatacaggtctgatcaacagtattacatctatgaactatgtgaagtcacacgtagcacctaatcacgattcatttaccgcccaacaaatgtccattttggtgtcatttttgtcattgggaactttttttggggctttaactgcaccatttatatctgattcgtatggcaggaagcctactatcattttcagtacaattttcatcttctctatcggaaattctttacaggtaggtgctggaggaatcacattattgattgtgggaagggtcatttcaggtatcggtataggcgcaatttcagcggttgttccattataccaagcagaagctacacataaatcattaagaggtgctattatttctacttaccaatgggccattacctggggcttgctcgtgtcaagtgcagtgtcgcaagggacacacgcaagaaacgacgcatcttcgtatcggattcccatagggttgcaatatgtctggtcgtcatttctcgctatcgggatgttctttctccctgagagtccacgctattacgttttgaaagacaagctagatgaagcagctaaatctttatcgtttttaagaggtgtaccagtccatgattctgggttactggaagaactagttgaaataaaggcaacatatgattacgaggcatcttttggttcttcgaacttcattgattgttttatttcaagtaaaagtagaccaaagcaaactctaaggatgtttacgggaattgcccttcaagcatttcaacaattttcaggtatcaactttatattttactacggtgtcaatttcttcaataagacaggagtcagtaatagttatctggtttcatttataacctatgctgttaatgttgtctttaatgttcctggtttgttttttgtggaattttttggtagacgtaaggtgctggttgttgggggtgttatcatgactatagccaactttattgtggccattgttgggtgttccttaaagactgtagcggccgcaaaagttatgatagcatttatatgtctattcatagctgccttttctgctacatggggtggtgttgtttgggttatttcagcagaactgtacccattgggtgtgagatctaaatgtacggctatatgcgctgctgctaactggcttgtaaactttatttgtgctttaattaccccttatattgtagatactgggtcgcatacatcatcattaggtgcaaaaatattcttcatttggggctccttaaatgcgatgggggtgatagttgtttacttgaccgtttatgaaacgaagggtttgacattagaagagattgatgaattatatattaagtcatccactggtgtcgtgtcaccaaaatttaataaagatattagggaacgcgcacttaaattccaatacgatcctttgcaaagattagaagacggaaagaacacttttgttgctaaaagaaataattttgacgatgaaacaccaagaaatgattttcgaaatacgatatcgggcgaaatagatcatagtcccaatcaaaaagaagttcattctatcccagaacgtgttgatattcctactagtacagaaattcttgaaagcccgaacaaaagtagtggtatgacagtccctgtgtcaccttctctgcaagacgttccaatcccgcaaacaacagagcctgctgaaattcgaaccaaatatgtggacctaggaaatgggcttggtcttaatacgtataatagagggcctccctcactctcaagcgactcaagcgaagattacacagaagatgaaataggcgggccctcatctcaaggcgaccaaagtaatagaagtactatgaatgatattaatgattatatggcacgtctcattcacagtacttctactgcaagtaacacgacagataagttctccggtaaccaaagtacccttcgttaccacacggcttcctcacattcggatacaactgaagaggacagcaatttgatggacctgggaaacgggcttgccttgaatgcttataacagaggtccaccttcaattttaatgaattccagtgatgaagaggcaaatggtggtgagacgtctgataatttgaacacagctcaagacttggctggtatgaaggaacgaatggcgcagtttgcgcagagctatattgacaagagaggcggtctggaacctgaaactcaatctaatattttgagcacttctctctccgtgatggctgacactaatgaacataataatgaaatcctccactcaagcgaagaaaacgccactaatcaacctgtaaatgaaaataatgatttgaaataa

SEQ ID NO: 18 - DNA, 2655 bp, Saccharomyces cerevisiae; identical to SEQ ID NO: 17
  source 1..2655: mol_type = other DNA

SEQ ID NO: 19 - DNA, 1581 bp, Candida albicans
  source 1..1581: mol_type = other DNA
atgagtgcaaatatccaagctcttatgaaaagctatgtcaattttgacgaacacacttctggttccgctgccagaggtattttaatcggtatgtttgctgcttttggtgggtttttgtttggttacgacactggtactatttctggtgtattgtctatggactacgttaaagccagattccccaacaacaaaaccgatttcacttctggtgaaagttcccttattgtctccattttatcagtcggtacttttgttggttccttgattgccccattgttttccgatagaattggtcgtagatggacattgattttatctactttgattgtttttaacttgggagttcttttacaaactgttgccactgaaaagaaattgcttattgcaggtagagccattgccggtactggtgttggtttaatttcatctgttattcctaattatatttcggaaacaacaccaaagtgggctagaggtgctgtcactgcttcataccaatggatgatcacctggggtcttttaattgctgcttgtgccaacaagggttcccaaggtagaaaagactctggttcctatagaatacccattggtattcaatttttgtgggcattgattttgggtattggtttcttgtttctcccagaaaccccaagatactgggtttccaagtctgaagaaactaaagctaaagattctttgagaagaattagaaacttgcctgttgatcacccagatttggtgctggaatacgatgacattaaagcaaactttgatttcgaatccaaatatgccacttcttcttggacccaagttttcaaaaacgttaacaaacaacaccacagattattcactggggttgccatccaagctttgcaacaacttactggtattaatttcatcttctactatggtactcaattcttcaagcgttctggtattgaagatcctttccttatccaacttgccactaatattgttaatgttggtatgactgtgccgggtattattttggttgaaacctggggtagaagaccattgttgatggccggtagtgttgttatggctgtctcccaattgattgttgccattgttggtgttgctgctagcagtcatgctgcaaatcaatgtttagttgctttcagttgtattttcattgctggtttcgcagcaacttggggacctctttgttgggctatttgtggtgaatcctttgctttgaacgtgagactgaaatcaatctccttgtgtaccgcaagtaactggctttggaatttcggtattggttatgctactccttatatggttgattcaggtaaagggaacgccgacttgggttcaaaggtgtttttcatttggggtggatgtaacgtcattggtgggttgtttgcatactttatggtttacgaaaccaagagtcttacattagaacaagttgatgaattgtacttgaaagttgatcacgcttggcaatctaaaggatttgttcctagtgtccacgcatttagagatgatggcgatattgagcacatctcttctgatggaaaagccgaaatggttgaagttgatgaaaattccgtttaa

SEQ ID NO: 20 - DNA, 129 bp, synthetic construct
  misc_feature 1..129: note = Synthetic construct
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 21 - RNA, 76 nt, Saccharomyces cerevisiae
  source 1..76: mol_type = unassigned RNA
tgcttaaggcctaaaacataccagatcgccacccgcgctttaatctggagaggtgaatacgaccacctaggccaaa

SEQ ID NO: 22 - DNA, 111 bp, Ostreococcus tauri
  source 1..111: mol_type = other DNA
cgcgcgtcgtgcgagtggctcgatcgatctcacgctcgatcgcgtctgagaacacatcgctggaacttgactcaggataatacctgcgtaaggaacgaccgcggcatcgcg

SEQ ID NO: 23 - DNA, 1362 bp, synthetic construct
  misc_feature 1..1362: note = Synthetic construct
  source 1..1362: mol_type = other DNA
tggctgcgtctggtgggaccgttgtatacgccggtagtggtttgcggggctacggcgagttggtcatcatcaaacacaacgagacctacgtgagtgcctacggtcacaaccgcaggctgctggtgcgggaagggcaacaggtcaaggtagggcaatcgattgccgagatgggctccacaggaaccgatcgggtgaagctgcacttcgagattcgccgccagggtaagcctgtcgatccactgcaatatttgccacgtcgctgaccgggagttcgcccgcccacatcatgtaggtgagcgggtccgggcgtgtccagcgggaaaggaatcgcccgggcttgagtcgaactcatgcaagggataacgacatggcactcaaaaaagaagggccggagtttgaccacgatgatgaagtgctcctcctggagcccggcatcatgctggacgagtcgtctgccgacgagcagccttctccccgggcaactccaaaagccaccacttccttctcttccaaacaacacaagcacatcgactacacgcgcgcgttggacgcaacgcagctgtatctcaacgaaatcggtttctcgcccctgttgacgcccgaagaggaagtccacttcgctcgtctggcgcagaagggcgatcccgctggtcggaagcggatgatcgagagcaacctgcggttggtggtgaagatcgcccggcgctatgtcaatcgcggactgtccctgctcgacctgatcgaggaaggcaacctaggcctgatccgcgccgtggagaagttcgatccggagcgcggattccggttctcgacctacgccacctggtggatccgccagaccatcgagcgggccatcatgaaccagacccggaccattcgcttgccgatccatgtggtcaaggagctcaacgtctacctgcgtgcggcgcgggaactgacccacaagctcgaccacgaaccttcacccgaagaaatcgccaacctgctggagaagccggtcgccgaggtcaagcgcatgctcggcctgaacgaacgggtgacttcggtagacgtctctcttggtccggactcggacaagaccctgctggatacgctcaccgacgatcgccccaccgatccgtgcgagctgctgcaggatgacgatctcagcgaaagcagctgacggaactcaccgacaagcagcgtgaggtggtgattcgccgcttcggcttgcgcggtcacgaaagcagcacgctggaagaggtcggccaggaaatcggcctgacccgcgagcgggttcgtcagatccaggtcgaggcgctgaagcgcctgcgggagattctggagaagaatggcctgtcgagtgacgcgctgttccagtga

SEQ ID NO: 24 - DNA, 29 bp, synthetic construct
  misc_feature 1..29: note = Synthetic construct
  misc_feature 13..15: note = n is a, c, g, or t
  misc_feature 18..20: note = n is a, c, g, or t
  source 1..29: mol_type = other DNA
tctagagaaagannngannntactagatg

SEQ ID NO: 25 - DNA, 18 bp, synthetic construct
  misc_feature 1..18: note = Synthetic construct
  source 1..18: mol_type = other DNA
cccgccgccaccatggag

SEQ ID NO: 26 - DNA, 13 bp, synthetic construct
  misc_feature 1..13: note = Synthetic construct
  source 1..13: mol_type = other DNA
tcacacaggaaag

SEQ ID NO: 27 - DNA, 979 bp, synthetic construct
  misc_feature 1..979: note = Synthetic construct
  source 1..979: mol_type = other DNA
tccctatcagtgatagagattgacatccctatcagtgatagagatactgagcactactagagaaagaggagaaatactagatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttata

(19) United States
(12) Patent Application Publication
(10) Pub. No.: US 2025/0035637 A1
(43) Pub. Date: Jan. 30, 2025

(54) GLUCOSE SENSORS AND METHODS OF USE THEREOF

(71) Applicant: BIOCAPITAL HOLDINGS, LLC, Houston, TX (US)

(72) Inventors: Raul CUERO RENGIFO, Cypress, TX (US); Laura Sanchez LONDONO, Manizales (CO)

(21) Appl. No.: 18/915,786

(22) Filed: Oct. 15, 2024

Related U.S. Application Data
(62) Division of application No. 17/311,106, filed on Jun. 4, 2021, now Pat. No. 12,146,883.
(60) Provisional application No. 62/775,994, filed on Dec. 6, 2018.

(51) Int. Cl. G01N 33/66; C07K 14/39; C12N 9/10; C12N 9/24; G01N 21/64

(52) U.S. Cl. CPC G01N 33/66; C07K 14/39; C12N 9/1051; C12N 9/2402; C12Y 204/01255; C12Y 302/01031; G01N 21/6428

(57) ABSTRACT

Described herein are glucose sensors. The sensors are composed of host cells incorporating DNA devices specifically designed to produce fluorescence when the cells come into contact with glucose from a patient sample. Once the fluorescence has been quantified, it can be correlated with the amount of glucose present in the sample. Also described herein are extracts from the host cells that can sense and measure glucose levels in a patient. The devices and extracts disclosed herein are inexpensive but sensitive and accurate enough for use in both home and clinical testing situations. The devices and extracts disclosed herein are also useful for diagnosis of diabetes, pre-diabetes, or other diseases associated with elevated glucose levels.

### CROSS REFERENCE TO RELATED APPLICATIONS

This application is a divisional application of U.S. nonprovisional application Ser. No. 17/311,106 filed on Jun. 4, 2021, which is a US national phase application of international application no. PCT/US2019/064470 filed on Dec. 4, 2019, which claims priority upon U.S. provisional application Ser. No. 62/775,994 filed on Dec. 6, 2018. This application is hereby incorporated by reference in its entirety.

### CROSS REFERENCE TO SEQUENCE LISTING

The instant application contains a Sequence Listing in ST.26 format entitled 930201-1111_Sequence_Listing.xml created on Oct. 14, 2024, and having a file size of 95,185 bytes. The content of the sequence listing is incorporated herein in its entirety.

### BACKGROUND

Diabetes is a degenerative disease caused by abnormal levels of glucose in the cell. These abnormal levels of glucose are not easily predictable and/or measurable. Current home use technologies to determine glucose levels in the blood are not always accurate and may not be able to determine extremely low levels of glucose (below 20 mg/dL). In type 1 diabetes, the body is unable to produce insulin. In type 2 diabetes, insulin is produced but the body destroys it or is unable to recognize it. Pre-diabetes, meanwhile, is a condition where blood glucose is higher than normal but not high enough to be considered type 2 diabetes. Prediabetes can often be addressed with lifestyle changes. Metabolic syndrome is a group of conditions occurring in the same individual including hypertension, high blood sugar, excess body fat around the waist, and high cholesterol. People with metabolic syndrome exhibit insulin resistance, as well.

Popular devices and methods for determining glucose levels employ enzyme reactions and use whole blood, plasma, or serum for samples. Enzymes used include glucose oxidase, hexokinase, and glucose dehydrogenase. The products of these enzymatic reactions and blood sugar can be determined using colorimetric or spectrophotometric assays, or by measuring electric current produced during the enzymatic reaction, as is the case for most commercially-available glucose meters. These methods can detect glucose in the range of 0-500 mg/dL for laboratory assays and 20-500 mg/dL for home assays and are becoming more sensitive, but falsely high readings can occur depending on environmental conditions and/or specific medical treatments a patient is undergoing. Use of home meters with incompatible strips as well as poor calibration of meters can also give unreliable results.

The cost of current methods for monitoring blood sugar levels is also an impediment and can range from $0.35 to $1 per test strip for home monitoring methods. Type 1 diabetics may test as often as four to ten times per day, making daily testing expensive. For clinical laboratories, glucose determinations can range from $3 to over $100 in the US and/or overseas. Diagnostic tests to detect the condition of diabetes in a previously-undiagnosed patient can range between $190 and $350. Furthermore, current methods require the use of lancing devices to generate blood samples; these devices can also be expensive and their use can cause pain in patients.

What is needed is an inexpensive and sensitive method to test glucose levels and diagnose diabetes and/or prediabetic conditions in patients. Ideally, the method would be non-invasive and not require the use of lancing devices.

### SUMMARY

Described herein are glucose sensors. The sensors are composed of host cells incorporating DNA devices specifically designed to produce fluorescence when the cells come into contact with glucose from a patient sample. Once the fluorescence has been quantified, it can be correlated with the amount of glucose present in the sample. Also described herein are extracts from the host cells that can sense and measure glucose levels in a patient. The devices and extracts disclosed herein are inexpensive but sensitive and accurate enough for use in both home and clinical testing situations. The devices and extracts disclosed herein are also useful for diagnosis of diabetes, pre-diabetes, or other diseases associated with elevated glucose levels.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a linear schematic of a vector described herein, wherein the vector has SEQ ID NO. 5.

FIG. 2 shows a circular schematic of a vector described herein, wherein the vector has SEQ ID NO. 5.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and appended claims, the singular forms “a,” “an,” and “the” include plural referents unless the context clearly dictates otherwise. Thus, for example, reference to “an isolated nucleic acid” includes mixtures of two or more such nucleic acids, and the like.

“Optional” or “optionally” means that the subsequently described event or circumstance can or cannot occur, and that the description includes instances where the event or circumstance occurs and instances where it does not. For example, the phrase “optionally includes a gene for a selective marker” means that the gene may or may not be present.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed that while specific reference of each various individual and collective combination and permutation of these compounds may not be explicitly disclosed, each is specifically contemplated and described herein. For example, if a yeast is disclosed and discussed and a number of different compatible yeast plasmids are discussed, each and every combination and permutation of yeast and yeast plasmid that is possible is specifically contemplated unless specifically indicated to the contrary. For example, if a class of molecules A, B, and C are disclosed as well as a class of molecules D, E, and F, and an example of a combination molecule A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if a variety of additional steps can be performed, it is understood that each of these additional steps can be performed with any specific embodiment or combination of embodiments of the disclosed methods, and that each such combination is specifically contemplated and should be considered disclosed.

References in the specification and concluding claims to parts by weight, of a particular element or component in a composition or article, denote the weight relationship between the element or component and any other elements or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### I. DNA Constructs

DNA constructs are provided herein for the production of SNF3, O-linked N-acetylglucosamine transferase (OGT), and O-linked N-acetylglucosamine-selective-N-acetyl-β-D-glucosaminidase (OGlcNa) for the production of devices and extracts that can be used to measure glucose levels in a patient sample. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning the two sequences so that the homology is at its highest level. Another way of calculating homology can be performed according to published algorithms (see Zuker, M., Science, 244:48-52, 1989; Jaeger et al., Proc. Natl. Acad. Sci. USA, 86:7706-7710, 1989; and Jaeger et al., Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least 70%, 75%, 80%, 85%, 90%, 95%, or 99% homology to a particular sequence wherein the variants are conservative or silent mutations. It is understood that any of the sequences described herein can be a variant or derivative having the homology values listed above.

In one aspect, a database such as, for example, GenBank, can be used to determine the sequences of genes and/or regulatory regions of interest, the species from which these elements originate, and related homologous sequences.

In one aspect, genes of interest can be incorporated into a DNA construct. In a further aspect, the DNA construct can be incorporated as part of a vector for transfection into microbial cells. In a still further aspect, the vector can be a plasmid, a phagemid, a cosmid, a yeast artificial chromosome, a bacterial artificial chromosome, a virus, a phage, or a transposon. In another aspect, the microorganisms are fungi or bacteria. In one aspect, the fungi are yeasts such as, for example, Saccharomyces cerevisiae. In another aspect, the bacteria are Escherichia coli.

Vectors capable of high levels of expression of recombinant genes and proteins are well known in the art. Vectors useful for the transformation of a variety of host cells are common and commercially available and include, for example, pWLneo, pSV2cat, pOG44, pXT1, pSG, pSVK3, pBSK, pBR322, pYES, pYES2, pBSKII, pUC, and pETDuet-1. The skilled practitioner will be able to choose a plasmid based on such factors as (a) the amount of nucleic acid (i.e., number of genes and other elements) to be inserted, (b) the host organism, (c) culture conditions for the host organism, and other related factors.

In one aspect, the DNA construct includes the following genetic components: (a) a gene that expresses SNF3, (b) a gene that expresses OGT, and (c) a gene that expresses OGlcNA.

In one aspect, the nucleic acids (e.g., genes that express the SNF3, OGT, and OGlcNA) used in the DNA constructs described herein can be amplified using polymerase chain reaction (PCR) prior to being ligated into a plasmid or other vector. Typically, PCR amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or gene. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein. In one aspect, disclosed herein are biological devices incorporating a gene that expresses SNF3. Without wishing to be bound by theory, SNF3 is a protein from yeasts and other organisms that has a high affinity glucose in the environment and that is further involved in glucose uptake; glucose at low concentrations stimulates SNF3 to regulate activity of genes encoding glucose transporters. In one aspect, SNF3 is a plasma membrane protein with a long C-terminal tail that extends into the cytoplasm. In some aspects, SNF3 may also respond to the presence of galactose in the environment.

In one aspect, the gene that expresses SNF3 is isolated from Pichia stipitis (also known as Scheffersomyces stipitis) and can be found in GenBank with GI number XM_001386369.1. In a further aspect, the gene that expresses SNF3 has SEQ ID NO. 1 or at least 70% homology thereto, at least 75% homology thereto, at least 80% homology thereto, at least 85% homology thereto, at least 90% homology thereto, or at least 95% homology thereto. In one aspect, the gene that expresses SNF3 is isolated from a yeast of one of the following genera: Babjeviella, Candida, Clavispora, Cyberlindnera, Debaryomyces, Hyphopichia, Kazachstania, Komagataella, Kuraishia, Lachancea, Lodderomyces, Metschnikowia, Meyerosyma, Naumovozyma, Pichia, Saccharomyces, Saccharomycesaceae, Saccharomycopsis, Scheffersomyces, Spathaspora, Vanderwaltozyma, or Wickerhamomyces. In another aspect, the gene that expresses SNF3 is isolated from one of the following strains of Saccharomyces cerevisiae: Y169, X55, KSD-Yc, SY14, BY4742, CEN.PK113-7D, YPS128, Y12, SK1, DBVPG6044, S288c, YJM1381, YJM1549, YJM1401, YJM1304, YJM1190, YJM1400, YJM1355, YJM1273, T63, HB S BILANCHER, HB C OMARUNUI, WA C MATES, WA C WAITAKEREROAD, T.52 5A, WA C KINGSMILL, WA C MATES, T78, HCNTHsf, T52.2H, T52.3C, YJM1439, YJM1342, YJM1479, YJM1434, YJM1389, YJM1388, YJM1248, or NSERVsf. In still another aspect, the gene that expresses SNF3 is isolated from one of the following strains of Candida albicans: SC5314-P0 or SC5314-GTH12. In an alternative aspect, the gene that expresses SNF3 is isolated from a plant such as, for example, Hordeum vulgare or Arabidopsis thaliana. In an alternative aspect, the gene that expresses SNF3 is isolated from a mammal such as, for example, a mouse. In still another aspect, the gene that expresses SNF3 has SEQ ID NO. 6, 7, 8, 9, 10, 11, 12, 13, 14, or 15.

Other sequences expressing SNF3 or related or homologous genes can be identified in a database such as, for example, GenBank. In one aspect, sequences useful herein include those with the GI numbers listed in Table 1.

TABLE 1
SNF3 Genes

| Source Organism | Sequence Description | GI Number |
|---|---|---|
| Scheffersomyces stipitis | high-affinity glucose | XM_001386379.1 |
| | transporter SNF3 | |
| Scheffersomyces stipitis | chromosome 8 sequence | CP000502.1 |
| Candida tanzawaensis | general substrate transporter | XM_020208803.1 |
| Spathaspora passalidarum | hypothetical protein | XM_007376130.1 |
| Debaryomyces hansenii | chromosome B sequence | CR382134.2 |
| Debaryomyces hansenii | genomic DNA | XM_457565.1 |
| Debaryomyces fabryi | hypothetical protein | XM_015611621.1 |
| Candida orthopsilosis | glucose and galactose sensor | XM_003868724.1 |
| Candida orthopsilosis | chromosome 3 sequence | HE681721.1 |
| Meyerosyma guilliermondii | hypothetical protein | XM_001486109.1 |
| Candida dubliniensis | glucose sensor (putative) | XM_002419355.1 |
| Candida dubliniensis | chromosome 3 sequence | FM992690.1 |
| Metschnikowia bicuspidata | hypothetical protein | XM_018856772.1 |
| Candida parapsilosis | genomic DNA | HE605208.1 |
| Candida auris | hypothetical protein | XM_018315448.1 |
| Lodderomyces elongisporus | hypothetical protein | XM_001526242.1 |
| Candida pseudohaemutonii | hypothetical protein | XM_024859329.1 |
| Candida duobushaemulonis | hypothetical protein | XM_025483142.1 |
| Candida albicans | chromosome 3 sequence | CP032018.1 |
| Candida viswanathii | high-affinity glucose | XM_026737497.1 |
| | transporter SNF3 | |
| Candida albicans | chromosome 3B sequence | CP025160.1 |
| Candida albicans | chromosome 3A sequence | CP025152.1 |
| Candida albicans | chromosome 3B sequence | CP025177.1 |
| Candida albicans | chromosome 3A sequence | CP025169.1 |
| Candida albicans | chromosome 3 sequence | CP017625.1 |
| Candida albicans | glucose sensor | XM_718080.1 |
| Candida tenuis | hypothetical protein | XM_006688525.1 |
| Clavispora lusitaniae | hypothetical protein | XM_002618452.1 |
| Candida viswanathii | high-affinity glucose | XM_026731331.1 |
| | transporter SNF3 | |
| Candida haemulonis | hypothetical protein | XM_025488849.1 |
| Candida tropicalis | hypothetical protein | XM_002547995.1 |
| Hyphopichia burtonii | general substrate transporter | XM_020218846.1 |
| | SNF3 | |
| Candida intermedia | chromosome II sequence | LT635765.1 |
| Candida intermedia | chromosome II sequence | LT635757.1 |
| Pichia sorbitophila | chromosome C sequence | FO082057.1 |
| Pichia sorbitophila | chromosome D sequence | FO082056.1 |
| Saccharomycetaceae sp. | chromosome IV sequence | CP006023.1 |
| “Ashbya aceri“ | | |
| Cyberlindnera fabianii | genomic DNA | LK052888.1 |
| Saccharomyces kudriavzevii | chromosome IV sequence | CP030965.1 |
| Lachancea lanzarotensis | genomic DNA | XM_022772444.1 |
| Wickerhamomyces ciferrii | high-affinity glucose | XM_011278392.1 |
| | transporter | |
| Wickerhamomyces anomalus | hypothetical protein | XM_019182027.1 |
| Babjeviella inositovora | hypothetical protein | XM_019129516.1 |
| Lachancea thermotolerans | genomic DNA | XM_002555927.1 |
| Lachancea thermotolerans | chromosome H sequence | CU928180.1 |
| Saccharomyces jurei | chromosome IV sequence | LT986465.1 |
| Naumovozyma castellii | hypothetical protein | XM_003676753.1 |
| Naumovozyma castellii | chromosome 5 sequence | HE576756.1 |
| Kuraishia capsulate | uncharacterized protein | XM_022605974.1 |
| Kazachstania naganishii | hypothetical protein | XM_022611574.1 |
| Saccharomyces paradoxus | chromosome IV sequence | CP020313.1 |
| Kazachstania naganishii | chromosome 2 sequence | HE978315.1 |
| Saccharomycopsis fibuligera | chromosome B5 sequence | CP012820.1 |
| Vanderwaltozyma polyspora | hypothetical protein | XM_001643718.1 |
| Saccharomyces eubayanus | chromosome IV sequence | CP030948.1 |
| Saccharomyces eubayanus | SNF3-like protein | XM_018364102.1 |
| Saccharomyces cerevisiae | chromosome 4 sequence | CP033473.1 |
| Saccharomyces cerevisiae | chromosome 4 sequence | CP033490.1 |
| Saccharomyces cerevisiae | chromosome 4 sequence | CP023998.1 |
| Saccharomyces cerevisiae | chromosome I sequence | CP029160.1 |
| Saccharomyces cerevisiae | chromosome IV sequence | CP026298.1 |
| Saccharomyces cerevisiae | chromosome IV sequence | CP022969.1 |
| Komagataella phaffii | chromosome 1 sequence | LT962476.1 |
| Saccharomyces cerevisiae | chromosome IV sequence | CP020211.1 |
| Saccharomyces cerevisiae | chromosome IV sequence | CP020194.1 |
| Saccharomyces cerevisiae | chromosome IV sequence | CP020177.1 |
| Saccharomyces cerevisiae | chromosome IV sequence | CP020143.1 |
| Saccharomyces cerevisiae | chromosome IV sequence | CP020126.1 |
| Saccharomyces cerevisiae | chromosome IV sequence | CP004718.2 |
| Pichia membranifaciens | hypothetical protein | XM_019160717.1 |
| Saccharomyces cerevisiae | chromosome IV sequence | CP004747.2 |
| Saccharomyces cerevisiae | chromosome IV sequence | CP004727.2 |
| Saccharomyces cerevisiae | chromosome IV sequence | CP004707.2 |
| Saccharomyces cerevisiae | chromosome IV sequence | CP004697.2 |
| Komagataella phaffii | chromosome 1 sequence | CP014715.1 |
| Komagataella phaffii | chromosome 1 sequence | CP014708.1 |
| Saccharomyces cerevisiae | chromosome IV sequence | CP004726.2 |
| Saccharomyces cerevisiae | chromosome IV sequence | CP004716.2 |
| Saccharomyces cerevisiae | chromosome IV sequence | CP004706.2 |
| Saccharomyces cerevisiae | chromosome IV sequence | CP008307.1 |
| Saccharomyces cerevisiae | chromosome IV sequence | CP008273.1 |
| Saccharomyces cerevisiae | chromosome IV sequence | CP008426.1 |
| Saccharomyces cerevisiae | chromosome IV sequence | CP008579.1 |
| Saccharomyces cerevisiae | chromosome IV sequence | CP008562.1 |
| Saccharomyces cerevisiae | chromosome IV sequence | CP008664.1 |
| Saccharomyces cerevisiae | chromosome IV sequence | CP008613.1 |
| Saccharomyces cerevisiae | chromosome IV sequence | CP008596.1 |
| Saccharomyces cerevisiae | chromosome IV sequence | CP007950.1 |
| Saccharomyces cerevisiae | chromosome IV sequence | CP007933.1 |
| Saccharomyces cerevisiae | chromosome IV sequence | CP007848.1 |
| Saccharomyces cerevisiae | chromosome IV sequence | CP007814.1 |
| Saccharomyces cerevisiae | chromosome IV sequence | CP004735.2 |
| Saccharomyces cerevisiae | chromosome IV sequence | CP004715.2 |
| Saccharomyces cerevisiae | chromosome IV sequence | CP004744.2 |
| Saccharomyces cerevisiae | chromosome IV sequence | CP004734.2 |
| Saccharomyces cerevisiae | chromosome IV sequence | CP004724.2 |
| Saccharomyces cerevisiae | chromosome IV sequence | CP004723.2 |
| Saccharomyces cerevisiae | chromosome IV sequence | CP004703.2 |
| Saccharomyces cerevisiae | chromosome IV sequence | CP008290.1 |
| Saccharomyces cerevisiae | chromosome IV sequence | CP007899.1 |

In one aspect, disclosed herein are biological devices incorporating a gene that expresses OGT, also known as O-linked N-acetylglucosamine transferase, O-GlcNAc transferase, and UDP-N-acetylglucosamine-peptide N-acetylglucosaminyltransferase. Without wishing to be bound by theory, OGT is a protein from a variety of organisms including birds, primates, rodents, carnivores, and other mammals that catalyzes the addition of a single N-acetylglucosamine to serine or threonine residues of intracellular proteins through an O-glycosidic linkage. OGT activity may compete with phosphorylation and reaction rate and specificity may be affected by steric and/or electrostatic effects or protein conformation and residue accessibility. OGT activity is implicated in a variety of functions in humans and animals. In one aspect, OGT is involved in insulin resistance in muscle and fat cells.

In one aspect, the gene that expresses OGT is isolated from Urocitellys parryii (commonly known as the arctic ground squirrel) and can be found in GenBank with GI number XM_02651267.1. In a further aspect, the gene that expresses OGT has SEQ ID NO. 2 or at least 70% homology thereto, at least 75% homology thereto, at least 80% homology thereto, at least 85% homology thereto, at least 90% homology thereto, or at least 95% homology thereto. In another aspect, the gene that expresses OGT is isolated from a human, a domestic or wild mammal, or a bird. In still another aspect, the gene that expresses OGT is isolated from a arctic ground squirrel, thirteen-lined ground squirrel, water buffalo, wild yak, grizzly bear, domestic cow, olive baboon, polar bear, gelada, sooty mangabey, drill, mountain lion, giant panda, crab-eating macaque, Angola colobus, domestic goat, rhesus macaque, domestic sheep, mouflon, southern pig-tailed macaque, golden snub-nosed monkey, alpine marmot, dingo, domestic horse, domestic dog, green monkey, domestic cat, white-tailed deer, leopard, European rabbit, red fox, Sumatran orangutan, gorilla, Siberian tiger, common marmoset, Weddell seal, Tibetan antelope, common chimpanzee, donkey, bonobo, human, aardvark, beluga whale, society finch white rhinoceros, gray mouse lemur, or black-capped squirrel monkey.

Other sequences expressing OGT or related or homologous genes can be identified in a database such as, for example, GenBank. In one aspect, sequences useful herein include those with the GI numbers listed in Table 2.

TABLE 2
OGT Genes

| Source Organism | Sequence Description | GI Number |
|---|---|---|
| Urocitellus parryii | O-linked N-acetylglucosamine transferase | XM_026412627.1 |
| Urocitellus parryii | O-linked N-acetylglucosamine transferase | XM_026412628.1 |
| Ictidomys tridecemlineatus | O-linked N-acetylglucosamine transferase | XM_021719915.1 |
| Ictidomys tridecemlineatus | O-linked N-acetylglucosamine transferase | XM_005339941.2 |
| Ictidomys tridecemlineatus | O-linked N-acetylglucosamine transferase | XM_005339942.2 |
| Bubalus bubalis | O-linked N-acetylglucosamine transferase | XM_006050820.2 |
| Bos mutus | O-linked N-acetylglucosamine transferase | XM_005900810.2 |
| Bubalus bubalis | O-linked N-acetylglucosamine transferase | NM_001290907.1 |
| Bos mutus | O-linked N-acetylglucosamine transferase | XM_005900811.2 |
| Ursus arctos horribilis | O-linked N-acetylglucosamine transferase | XM_026485785.1 |
| Bos taurus | O-linked N-acetylglucosamine transferase | XM_005228027.4 |
| Papio anubis | O-linked N-acetylglucosamine transferase | XM_003917862.3 |
| Ursus maritimus | O-linked N-acetylglucosamine transferase | XM_008712243.1 |
| Ursus arctos horribilis | O-linked N-acetylglucosamine transferase | XM_026485786.1 |
| Papio anubis | O-linked N-acetylglucosamine transferase | XM_003917863.4 |
| Ursus maritimus | O-linked N-acetylglucosamine transferase | XM_008712244.1 |
| Bos taurus | O-linked N-acetylglucosamine transferase | NM_001098070.2 |
| Bos taurus | O-linked N-acetylglucosamine transferase | BC140542.1 |
| Theropithecus gelada | O-linked N-acetylglucosamine transferase | XM_025371879.1 |
| Cercocebus atys | O-linked N-acetylglucosamine transferase | XM_012060760.1 |
| Theropithecus gelada | O-linked N-acetylglucosamine transferase | XM_025371880.1 |
| Cercocebus atys | O-linked N-acetylglucosamine transferase | XM_012060761.1 |
| Mandrillus leucophaeus | O-linked N-acetylglucosamine transferase | XM_011992070.1 |
| Mandrillus leucophaeus | O-linked N-acetylglucosamine transferase | XM_011992071.1 |
| Puma concolor | O-linked N-acetylglucosamine transferase | XM_025933892.1 |
| Ailuropoda melanoleuca | O-linked N-acetylglucosamine transferase | XM_002930577.3 |
| Macaca fascicularis | O-linked N-acetylglucosamine transferase | XM_005593919.1 |
| Colobus angolensis palliatus | O-linked N-acetylglucosamine transferase | XM_011943084.1 |
| Puma concolor | O-linked N-acetylglucosamine transferase | XM_025933893.1 |
| Ailuropoda melanoleuca | O-linked N-acetylglucosamine transferase | XM_002930576.3 |
| Macaca fascicularis | O-linked N-acetylglucosamine transferase | XM_005593920.2 |
| Capra hircus | O-linked N-acetylglucosamine transferase | XM_013976396.2 |
| Macaca mulatta | O-linked N-acetylglucosamine transferase | XM_015127666.1 |
| Ovis aries | O-linked N-acetylglucosamine transferase | XM_004022178.3 |
| Ovis aries musimon | O-linked N-acetylglucosamine transferase | XM_012142301.2 |
| Macaca nemestrina | O-linked N-acetylglucosamine transferase | XM_011732741.1 |
| Colobus angolensis palliatus | O-linked N-acetylglucosamine transferase | XM_011943085.1 |
| Rhinopithecus roxellana | O-linked N-acetylglucosamine transferase | XM_010366585.1 |
| Macaca nemestrina | O-linked N-acetylglucosamine transferase | XM_011732742.2 |
| Capra hircus | O-linked N-acetylglucosamine transferase | XM_013976398.2 |
| Marmota marmota marmota | UDP-N-acetylglucosamine-peptide N- | XR 001502441.1 |
| | acetylglucosaminetransferase | |
| Macaca mulatta | O-linked N-acetylglucosamine transferase | XM_015127667.1 |
| Ovis aries | O-linked N-acetylglucosamine transferase | XM_004022177.3 |
| Ovis aries musimon | O-linked N-acetylglucosamine transferase | XM_012142302.2 |
| Canis lupus dingo | O-linked N-acetylglucosamine transferase | XM_025466166.1 |
| Equus caballus | O-linked N-acetylglucosamine transferase | XM_001493372.5 |
| Canis lupus familiaris | O-linked N-acetylglucosamine transferase | XM_844299.5 |
| Rhinopithecus roxellana | O-linked N-acetylglucosamine transferase | XM_010366586.1 |
| Chlorocebus sabaeus | O-linked N-acetylglucosamine transferase | XM_007992035.1 |
| Canis lupus dingo | O-linked N-acetylglucosamine transferase | XM_025466167.1 |
| Canis lupus familiaris | O-linked N-acetylglucosamine transferase | XM_538075.6 |
| Chlorocebus sabaeus | O-linked N-acetylglucosamine transferase | XM_007992036.1 |
| Equus caballus | O-linked N-acetylglucosamine transferase | XM_005614262.3 |
| Felis catus | O-linked N-acetylglucosamine transferase | XM_004000621.5 |
| Odocoileus virgianus texanus | O-linked N-acetylglucosamine transferase | XM_020882917.1 |
| Odocoileus virgianus texanus | O-linked N-acetylglucosamine transferase | XM_020882916.1 |
| Panthera pardus | O-linked N-acetylglucosamine transferase | XM_019428710.1 |
| Oryctolagus cuniculus | O-linked N-acetylglucosamine transferase | XM_002720103.3 |
| Equus caballus | O-linked N-acetylglucosamine transferase | XM_001493388.5 |
| Felix catus | O-linked N-acetylglucosamine transferase | XM_004000622.5 |
| Odocoileus virgianus texanus | O-linked N-acetylglucosamine transferase | XM_020882919.1 |
| Odocoileus virgianus texanus | O-linked N-acetylglucosamine transferase | XM_020882918.1 |
| Panthera pardus | O-linked N-acetylglucosamine transferase | XM_019428712.1 |
| Oryctolagus cuniculus | O-linked N-acetylglucosamine transferase | XM_002720102.3 |
| Vulpes vulpes | O-linked N-acetylglucosamine transferase | XM_025982889.1 |
| Pongo abelii | O-linked N-acetylglucosamine transferase | XM_024240358.1 |
| Gorilla gorilla gorilla | O-linked N-acetylglucosamine transferase | XM_004064365.2 |
| Panthera tigris altaica | O-linked N-acetylglucosamine transferase | XM_007089115.2 |
| Callithrix jacchus | O-linked N-acetylglucosamine transferase | XM_002762977.2 |
| Leptonychotes weddellii | O-linked N-acetylglucosamine transferase | XM_006749440.1 |
| Pantholops hodgsonii | O-linked N-acetylglucosamine transferase | XM_005954790.1 |
| Vulpes vulpes | O-linked N-acetylglucosamine transferase | XM_025982890.1 |
| Pongo abelii | O-linked N-acetylglucosamine transferase | XM_024240359.1 |
| Gorilla gorilla gorilla | O-linked N-acetylglucosamine transferase | XM_004064366.2 |
| Callithrix jacchus | O-linked N-acetylglucosamine transferase | XM_002762978.3 |
| Panthera tigris altaica | O-linked N-acetylglucosamine transferase | XM_007089116.2 |
| Leptonychotes weddellii | O-linked N-acetylglucosamine transferase | XM_006749441.1 |
| Pantholops hodgsonii | O-linked N-acetylglucosamine transferase | XM_005954791.1 |
| Pan troglodytes | O-linked N-acetylglucosamine transferase | XM_016943032.2 |
| Equus asinus | O-linked N-acetylglucosamine transferase | XM_014837684.1 |
| Pan paniscus | O-linked N-acetylglucosamine transferase | XM_003820131.2 |
| Homo sapiens | genomic DNA | AL833085.2 |
| Pan troglodytes | O-linked N-acetylglucosamine transferase | XM_016943033.1 |
| Pan paniscus | O-linked N-acetylglucosamine transferase | XM_003820132.2 |
| Equus asinus | O-linked N-acetylglucosamine transferase | XM_014837686.1 |
| Orycteropus afer afer | O-linked N-acetylglucosamine transferase | XM_007959000.1 |
| Homo sapiens | O-linked N-acetylglucosamine transferase | NM_181672.2 |
| Pongo abelii | O-linked N-acetylglucosamine transferase | NM_001133824.1 |
| Homo sapiens | genomic DNA | BX537844.1 |
| Homo sapiens | O-linked N-acetylglucosamine transferase | BC038180.1 |
| Equus asinus | O-linked N-acetylglucosamine transferase | XM_014837685.1 |
| Orycteropus afer afer | O-linked N-acetylglucosamine transferase | XM_007959001.1 |
| Homo sapiens | O-linked N-acetylglucosamine transferase | NM_181673.2 |
| Homo sapiens | O-linked N-acetylglucosamine transferase | BC014434.1 |
| Delphinapterus leucas | O-linked N-acetylglucosamine transferase | XM_022560995.1 |
| Lonchura striata domestica | O-linked N-acetylglucosamine transferase | XM_021534535.1 |
| Ceratotherium simum simum | O-linked N-acetylglucosamine transferase | XM_014794874.1 |
| Ceratotherium simum simum | O-linked N-acetylglucosamine transferase | XM_004439842.2 |
| Microcebus murinus | O-linked N-acetylglucosamine transferase | XM_012736183.1 |
| Saimiri boliviensis boliviensis | O-linked N-acetylglucosamine transferase | XM_003943099.2 |

In one aspect, disclosed herein are biological devices incorporating a gene that expresses OGlcNA, also known as O-GlcNAcase or O-GlcNAc-selective-N-acetyl-β-D-glucosaminidase, where GlcNAc is N-acetyl glucosamine and the prefix “O” indicates a linkage to a protein through an oxygen atom in an amino acid side chain. Without wishing to be bound by theory, glucosamine-induced insulin resistance is associated with an increase in or excess of proteins modified with O-GlcNAc residues, although O-GlcNAc modification is a common (though transient) post-translational modification of many cytoplasmic proteins. The gene encoding OGlcNA is also known as MEGA5 or meningioma expressed antigen 5 (hyaluronidase) and some variants in this gene are associated with an increased risk or precedence of type 2 diabetes in human populations.

In one aspect, the gene that expresses OGlcNA is isolated from Rattus norvegicus (commonly known as the brown rat) and can be found in GenBank with GI number NM_131904.1. In a further aspect, the gene that expresses OGlcNA has SEQ ID NO. 3 or at least 70% homology thereto, at least 75% homology thereto, at least 80% homology thereto, at least 85% homology thereto, at least 90% homology thereto, or at least 95% homology thereto. In another aspect, the gene that expresses OGlcNA is isolated from a rodent, carnivorous mammal, shrew, cetacean, pinniped, marsupial, primate, or other mammal. In still another aspect, the gene that expresses OGlcNA is isolated from a brown rat, alpine marmot, wild boar, arctic ground squirrel, thirteen-lined ground squirrel, domestic horse, Przewalski's horse, house mouse, European hedgehog, leopard, white rhinoceros, sea otter, Ord's kangaroo rat, North American beaver, Ryukyu mouse, Siberian tiger, Chinese tree shrew, cheetah, donkey, ferret, Gairdner's shrewmouse, naked mole-rat, domestic cat, Mongolian gerbil, beluga whale, olive baboon, Coquerel's sifaka, common bottlenose dolphin, Egyptian fruit bat, minke whale, gelada, Chinese rufous horseshoe bat, orca, dingo, northern fur seal, domestic dog, Sunda pangolin, common vampire bat, Pacific white-sided dolphin, gorilla, Sumatran orangutan, sperm whale, Ugandan red colobus, black snub-nosed monkey, drill, Bactrian camel, baiji, mouflon, Tibetan antelope, prairie vole, black flying fox, Florida manatee, large flying fox, African bush elephant, white-tailed deer, giant panda, green monkey, common degu, common shrew, Damara mole-rat, black-capped squirrel monkey, Tasmanian devil, great roundleaf bat, narrow-ridged finless porpoise, spalax, rhesus macaque, dromedary, golden snub-nosed monkey, koala, water buffalo, gray short-tailed opossum, Pacific walrus, common marmoset, southern pig-tailed macaque, common chimpanzee, Hawaiian monk seal, crab-eating macaque, polar bear, wild Bactrian camel, grizzly bear, gray mouse lemur, northern white-cheeked gibbon, or bonobo.

Other sequences expressing OGlcNA or related or homologous genes can be identified in a database such as, for example, GenBank. In one aspect, sequences useful herein include those with the GI numbers listed in Table 3.

TABLE 3
OGlcNA Genes

| Source Organism | Sequence Description | GI Number |
|---|---|---|
| Rattus norvegicus | O-GlcNAcase | NM_131904.1 |
| Rattus norvegicus | meningioma expressed antigen 5 | XM_017588708.1 |
| Marmota marmota marmota | meningioma expressed antigen 5 | XM_015494690.1 |
| Sus scrofa | meningioma expressed antigen 5 | XM_003483548.4 |
| Urocitellus parryii | O-GlcNAcase | XM_026407874.1 |
| Ictidomys tridecemlineatus | O-GlcNAcase | XM_013363728.2 |
| Equus caballus | meningioma expressed antigen 5 | XM_023640514.1 |
| Equus caballus | meningioma expressed antigen 5 | XM_001499535.5 |
| Equus przewalskii | meningioma expressed antigen 5 | XM_008520873.1 |
| Mus musculus | cytosolic beta-N- | AF132214.1 |
| | acetylglucosaminidase | |
| Erinaceus europaeus | meningioma expressed antigen 5 | XM_007533846.2 |
| Panthera pardus | meningioma expressed antigen 5 | XM_019459219.1 |
| Ceratotherium simum simum | meningioma expressed antigen 5 | XM_004427981.2 |
| Mus musculus | meningioma expressed antigen 5 | BC054821.1 |
| Enhydra lutris kenyoni | meningioma expressed antigen 5 | XM_022499195.1 |
| Enhydra lutris kenyoni | meningioma expressed antigen 5 | XM_022499194.1 |
| Dipodomys ordii | meningioma expressed antigen 5 | XM_013017723.1 |
| Castor canadensis | meningioma expressed antigen 5 | XM_020171238.1 |
| Mus caroli | meningioma expressed antigen 5 | XM_021151375.1 |
| Mus caroli | meningioma expressed antigen 5 | XM_021151374.1 |
| Mus caroli | meningioma expressed antigen 5 | XM_021151373.1 |
| Panthera tigris altaica | meningioma expressed antigen 5 | XM_007080283.1 |
| Mus musculus | meningioma expressed antigen 5 | NM_023799.4 |
| Tupaia chinensis | meningioma expressed antigen 5 | XM_006166334.2 |
| Acinonyx jubatus | O-GlcNAcase | XM_027062915.1 |
| Equus asinus | meningioma expressed antigen 5 | XM_014837652.1 |
| Mustela putorius furo | meningioma expressed antigen 5 | XM_004749434.2 |
| Mustela putorius furo | meningioma expressed antigen 5 | XM_004749433.2 |
| Mus musculus | hypothetical protein | AK129188.1 |
| Mus pahari | meningioma expressed antigen 5 | XM_021187683.1 |
| Heterocephalus glaber | meningioma expressed antigen 5 | XM_004866368.3 |
| Felis catus | meningioma expressed antigen 5 | XM_003994344.5 |
| Meriones unguiculatus | meningioma expressed antigen 5 | XM_021649955.1 |
| Delphinapterus leucas | meningioma expressed antigen 5 | XM_022568972.1 |
| Papio anubis | meningioma expressed antigen 5 | XM_003904159.3 |
| Propithecus coquereli | meningioma expressed antigen 5 | XM_012661710.1 |
| Tursiops truncates | meningioma expressed antigen 5 | XM_019939852.1 |
| Rousettus aegyptiacus | meningioma expressed antigen 5 | XM_016120014.1 |
| Balaenoptera acutorostrata | meningioma expressed antigen 5 | XM_007187305.1 |
| scammoni | | |
| Theropilhecus gelada | O-GlcNAcase | XM_025397972.1 |
| Rhinolophus sinicus | meningioma expressed antigen 5 | XM_019724961.1 |
| Orcinus orca | meningioma expressed antigen 5 | XM_012532559.1 |
| Orcinus orca | meningioma expressed antigen 5 | XM_004268486.2 |
| Canis lupus dingo | O-GlcNAcase | XM_025466984.1 |
| Callorhinus ursinus | O-GlcNAcase | XM_025880995.1 |
| Canis lupus famialiaris | meningioma expressed antigen 5 | XM_534996.6 |
| Manis javanica | meningioma expressed antigen 5 | XM_017674347.1 |
| Desmodus rotundus | meningioma expressed antigen 5 | XM_024555707.1 |
| Lagenorhynchus obliquidens | O-GlcNAcase | XM_027112366.1 |
| Gordla gorilla gorilla | meningioma expressed antigen 5 | XM_004049990.2 |
| Pongo abelii | meningioma expressed antigen 5 | XM_009245728.2 |
| Physeter catodon | meningioma expressed antigen 5 | XM_007116100.2 |
| Piliocolobus tephrosceles | O-GlcNAcase | XM_023206465.1 |
| Rhinopithecus bieti | meningioma expressed antigen 5 | XM_017851728.1 |
| Mandrillus leucophaeus | meningioma expressed antigen 5 | XM_011977442.1 |
| Camelus bactrianus | meningioma expressed antigen 5 | XM_010971007.1 |
| Lipotes vexilifer | meningioma expressed antigen 5 | XM_007470370.1 |
| Ovis aries musimon | meningioma expressed antigen 5 | XM_012119661.2 |
| Pantholops hodgsonii | meningioma expressed antigen 5 | XM_005980709.1 |
| Microtus ochrogaster | O-GlcNAcase | XM_005352373.3 |
| Pteropus alecto | O-GlcNAcase | XM_006925850.3 |
| Trichechus manatus latirostris | meningioma expressed antigen 5 | XM_004370059.2 |
| Pteropus vampyrus | meningioma expressed antigen 5 | XM_011378888.2 |
| Loxodonta africana | meningioma expressed antigen 5 | XM_003409163.3 |
| Odocoileus virginianus texanus | meningioma expressed antigen 5 | XM_020897815.1 |
| Odocoileus virginianus texanus | meningioma expressed antigen 5 | XM_020897813.1 |
| Ailuropoda melanoleuca | meningioma expressed antigen 5 | XM_011236048.2 |
| Ailuropoda melanoleuca | meningioma expressed antigen 5 | XM_002913873.3 |
| Chlorocebus sabaeus | meningioma expressed antigen 5 | XM_007963927.1 |
| Octodon degus | meningioma expressed antigen 5 | XM_004631450.2 |
| Odocoileus virginianus texanus | meningioma expressed antigen 5 | XM_020897814.1 |
| Sorex araneus | meningioma expressed antigen 5 | XM_004616334.2 |
| Fukomys damarensis | meningioma expressed antigen 5 | XM_010626504.1 |
| Saimiri boliviensis boliviensis | meningioma expressed antigen 5 | XM_003922263.2 |
| Sarcophilus harrisii | meningioma expressed antigen 5 | XM_003755444.3 |
| Hipposideros armiger | meningioma expressed antigen 5 | XM_019636367.1 |
| Neophocaena asiaeorientalis | O-GlcNAcase | XM_024734837.1 |
| asiaeorientalis | | |
| Nannospalax galili | meningioma expressed antigen 5 | XM_008845612.2 |
| Macaca mulatta | meningioma expressed antigen 5 | XM_015148051.1 |
| Camelus dromedarius | meningioma expressed antigen 5 | XM_010974469.1 |
| Rhinopithecus roxellana | meningioma expressed antigen 5 | XM_010356139.1 |
| Phascolarctos cinereus | meningioma expressed antigen 5 | XM_020967113.1 |
| Bubalus bubalis | O-GlcNAcase | NM_001290956.1 |
| Monodelphis domestica | meningioma expressed antigen 5 | XM_001369640.3 |
| Odobenus rosmarus divergens | meningioma expressed antigen 5 | XM_012562921.1 |
| Odobenus rosmarus divergens | meningioma expressed antigen 5 | XM_004401951.1 |
| Callithrix jacchus | meningioma expressed antigen 5 | XM_002756542.3 |
| Macaca nemestrina | O-GlcNAcase | XM_011735546.2 |
| Pan trodlogytes | meningioma expressed antigen 5 | XM_507996.7 |
| Neomonachus schauinslandi | meningioma expressed antigen 5 | XM_021700283.1 |
| Macaca fascicularis | meningioma expressed antigen 5 | XM_005566254.2 |
| Ursus maritimus | meningioma expressed antigen 5 | XM_008709033.1 |
| Equus caballus | meningioma expressed antigen 5 | XM_023640519.1 |
| Castor canadensis | meningioma expressed antigen 5 | XM_020171236.1 |
| Camelus ferus | meningioma expressed antigen 5 | XM_006183010.2 |
| Ursus arctos horribilis | O-GlcNAcase | XM_026493941.1 |
| Microcebus murinus | meningioma expressed antigen 5 | XM_012769426.2 |
| Microcebus murinus | meningioma expressed antigen 5 | XM_020280663.1 |
| Nomascus leucogenys | meningioma expressed antigen 5 | XM_003255376.2 |
| Pan paniscus | O-GlcNAcase | XM_003825499.2 |

In another aspect, said construct further includes a promoter, a terminator or stop sequence, a gene that confers resistance to an antibiotic (a “selective marker”), a reporter protein, or a combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In one aspect, the coding sequence to be controlled is located 3′ to the promoter. In another aspect, the promoter is derived from a native gene. In an alternative aspect, the promoter is composed of multiple elements derived from different genes and/or promoters. A promoter can be assembled from elements found in nature, from artificial or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter may also be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, a glucose promoter, and GAL1 promoter. In one aspect, the promoter is a glucose promoter. In a further aspect, the glucose promoter has SEQ ID NO. 16, 17, 18, or 19 or at least 70% homology thereto, at least 75% homology thereto, at least 80% homology thereto, at least 85% homology thereto, at least 90% homology thereto, or at least 95% homology thereto. In another aspect, the promoter is the native GAL1 promoter found in the plasmid pYES2. Variants of these promoters are also contemplated. The skilled artisan will be able to use site-directed mutagenesis and/or other mutagenesis techniques to modify the promoters to promote more efficient function. The promoter may be positioned, for example, at about 10-100 nucleotides from a ribosomal binding site. In another aspect, the promoter is positioned before the gene that expresses SNF3, OGT, OGlcNA, or a combination thereof. In an alternative aspect, several different promoters can be used in the same DNA construct.

In one aspect, the promoter is a GAL1 promoter. In another aspect, the GAL1 promoter is native to the plasmid used to create the vector. In another aspect, a GAL1 promoter is positioned before any or all genetic components present in the device. In another aspect, the promoter is a GAL1 promoter obtained from or native to the pYES2 plasmid.

In another aspect, the regulatory sequence is a terminator or stop sequence. As used herein, a “terminator” is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is a T7 terminator. In an alternative aspect, the terminator is a CYC1 terminator obtained from or native to the pYES2 plasmid. In still another aspect, the DNA construct can include multiple terminators. In one aspect, the terminator is native to the vector in which the DNA construct is incorporated. In an alternative aspect, a terminator is positioned after each gene of interest in the 5′ to 3′ direction. In one aspect, the terminator has SEQ ID NO. 20 or at least 70% homology thereto, at least 75% homology thereto, at least 80% homology thereto, at least 85% homology thereto, at least 90% homology thereto, or at least 95% homology thereto.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In another aspect, the vector contains one or more ribosomal binding sites. As used herein, a “ribosomal binding site” is a sequence of nucleotides located 5′ to the start codon of an mRNA that recruits a ribosome to initiate protein translation. In one aspect, the ribosomal binding site can be positioned before any or all genes in a DNA construct, or a before a subset of genes in a DNA construct. In one aspect, the ribosomal binding site has SEQ ID NO. 24, 25, or 26, or about 70% homology thereto, about 75% homology thereto, about 80% homology thereto, about 85% homology thereto, about 90% homology thereto, or about 95% homology thereto.

In some aspects, the vector further includes a riboswitch. In one aspect, the riboswitch has SEQ ID NO. 21, 22, or 23, or about 70% homology thereto, about 75% homology thereto, about 80% homology thereto, about 85% homology thereto, about 90% homology thereto, or about 95% homology thereto. As used herein, a “riboswitch” is a regulatory segment of an mRNA molecule that binds a small molecule such as, for example, glucose, thereby resulting in a change in production of the proteins encoded in the mRNA.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for multiple restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BsaBI, NotI, XhoI, SphI, XbaI, ApaI, SalI, ClaI, EcoRV, PstI, SmaI, SpeI, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior to insertion into the plasmid. In a further aspect, a DNA ligase enzyme can be used to assist in the ligation process.

In another aspect, the ligation mix may be incubated in an electromagnetic chamber. In one aspect, this incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and are commercially available. Such vectors include, but are not limited to, pWLneo, pSV2cat, pOG44, pXT1, pSG, pSVK3, pBSK, pBR322, pYES, pYES2, pBSKII, pUC, pUC19, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copies per host cell and will contain selectable markers (such as genes for antibiotic resistance) to allow the skilled artisan to select host cells that have been successfully transfected with the plasmids (for example, by growing the host cells in a medium containing the antibiotic).

In one aspect, the vector encodes a selective marker. In a further aspect, the selective marker is a gene that confers resistance to an antibiotic. In certain aspects, during fermentation of host cells transformed with the vector, the cells are contacted with the antibiotic. For example, the antibiotic may be included in the culture medium. Cells that have not successfully been transformed cannot survive in the presence of the antibiotic; only cells containing the vector that confers antibiotic resistance can survive. Optimally, only cells containing the vector to be expressed will be cultured, as this will result in the highest production efficiency of the desired gene products (e.g., proteins). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isoniazid, methicillin, oxacillin, vancomycin, streptomycin, quinolones, rifampin, rifampicin, sulfonamides, cephalothin, erythromycin, gentamycin, penicillin, other commonly-used antibiotics, or a combination thereof.

In certain aspects, the DNA construct can include a gene that expresses a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is a yellow fluorescent protein and the gene that expresses the reporter protein has SEQ ID NO. 27 or at least 70% homology thereto, at least 75% homology thereto, at least 80% homology thereto, at least 85% homology thereto, at least 90% homology thereto, or at least 95% homology thereto. In another aspect, the reporter protein is a green fluorescent protein and the gene that expresses the reporter protein has SEQ ID NO. 4 or at least 70% homology thereto, at least 75% homology thereto, at least 80% homology thereto, at least 85% homology thereto, at least 90% homology thereto, or at least 95% homology thereto. In one aspect, the green fluorescent protein is enhanced green fluorescent protein (EGFP).

The amount of fluorescence that is produced by the biological device can be correlated to the amount of DNA incorporated into the microbial host cells. The fluorescence produced by the device can be detected and quantified using techniques known in the art. For example, spectrofluorometers are typically used to measure fluorescence. The Examples provide exemplary procedures for measuring the amount of fluorescence produced as a result of the expression of DNA.

In one aspect, the construct includes the following genetic components: (a) a gene that expresses SNF3, (b) a gene that expresses OGT, and (c) a gene that expresses OGlcNA. In another aspect, the construct includes the following genetic components: (a) a gene that expresses SNF3, (b) a gene that expresses OGT, (c) a gene that expresses OGlcNA, and (d) a gene that expresses a reporter protein.

In another aspect, the DNA construct has the following genetic components: (1) one or more promoters, (2) a gene that expresses SNF3, (3) a gene that expresses OGT, (4) a gene that expresses OGlcNA, (5) a gene that expresses a reporter protein, and (6) one or more terminators or stop sequences.

In one aspect, the construct includes from 5′ to 3′ the following genetic components in the following order: (a) a gene that expresses SNF3, (b) a gene that expresses OGT, and (c) a gene that expresses OGlcNA. In another aspect, the construct includes from 5′ to 3′ the following genetic components in the following order: (a) a gene that expresses SNF3, (b) a gene that expresses OGT, (c) a gene that expresses OGlcNA, and (d) a gene that expresses a reporter protein.

In one aspect, the construct is a pYES2 or pBKSII plasmid having from 5′ to 3′ the following genetic components in the following order: (a) a gene that expresses SNF3, (b) a gene that expresses OGT, and (c) a gene that expresses OGlcNA. In another aspect, the construct is a pYES2 or pBKSII plasmid having from 5′ to 3′ the following genetic components in the following order: (a) a gene that expresses SNF3, (b) a gene that expresses OGT, (c) a gene that expresses OGlcNA, and (d) a gene that expresses a reporter protein.

In another aspect, the construct comprises from 5′ to 3′ the following genetic components in the following order: (a) a gene that expresses SNF3, (b) a CYC1 terminator, (c) a GAL1 promoter, (d) a gene that expresses OGT, (e) a CYC1 terminator, (f) a GAL1 promoter, (g) a gene that expresses OGlcNA, (h) a CYC1 terminator, (i) a GAL1 promoter, and (j) a gene that expresses a reporter protein.

In another aspect, the construct comprises from 5′ to 3′ the following genetic components in the following order: (a) a gene that expresses SNF3 having SEQ ID NO. 1 or at least 70% homology thereto, (b) a CYC1 terminator, (c) a GAL1 promoter, (d) a gene that expresses OGT having SEQ ID NO. 2 or at least 70% homology thereto, (e) a CYC1 terminator, (f) a GAL1 promoter, (g) a gene that expresses OGlcNA having SEQ ID NO. 3 or at least 70% homology thereto, (h) a CYC1 terminator, (i) a GAL1 promoter, and (j) a gene that expresses a reporter protein having SEQ ID NO. 4 or at least 70% homology thereto.

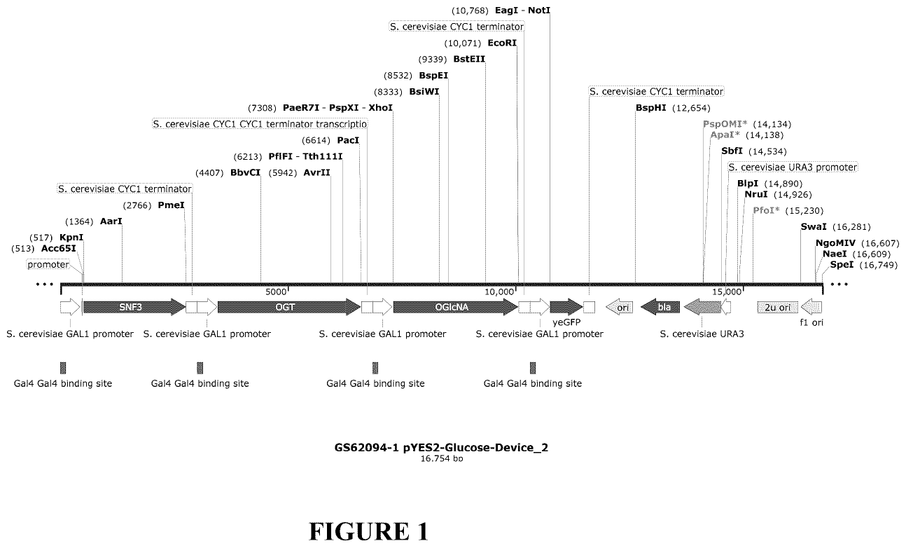

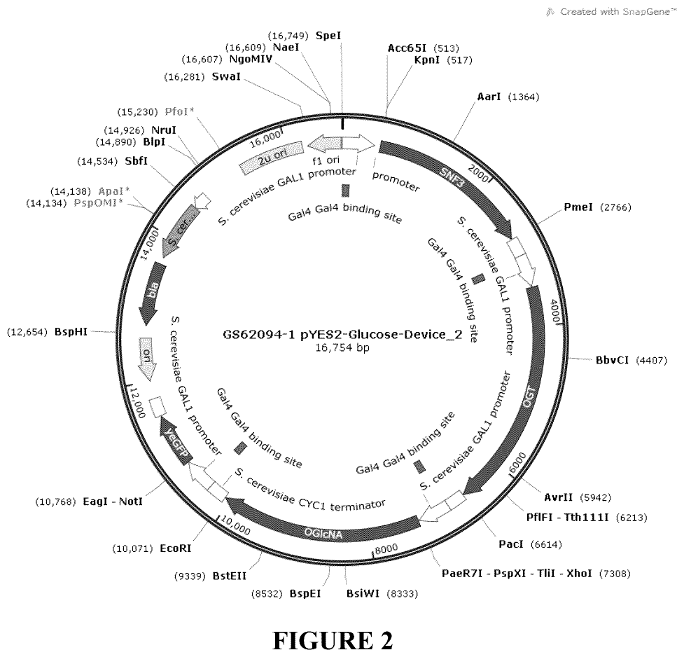

In another aspect, the DNA construct has SEQ ID NO. 5 or at least 70% homology thereto, at least 75% homology thereto, at least 80% homology thereto, at least 85% homology thereto, at least 90% homology thereto, or at least 95% homology thereto. FIGS. 1 and 2 provide non-limiting examples of DNA constructs described herein.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios such as, for example, 1:1, 1:2, 1:3, 1:4, and up to 1:5. In one aspect, the ratio of the backbone plasmid to synthetic insert is 1:4. After the vector comprising the DNA construct has been produced, the resulting vector can be incorporated into the host cells using the methods described below.

### II. Biological Devices

In one aspect, a “biological device” is formed when a microbial cell is transfected with the DNA construct described herein. The biological devices are generally composed of microbial host cells, where the host cells are transformed with a DNA construct as described herein.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce SNF3, OGT, and OGlcNA, devices and extracts that can be used to measure glucose levels. “Heterologous” genes and proteins are genes and proteins that have been experimentally inserted into a cell that are not normally expressed by that cell. A heterologous gene may be cloned or derived from a different cell type or species than the recipient cell or organism. Heterologous genes may be introduced into cells by transfection or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed for only a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the Examples.

The host cells as referred to herein include their progeny, which are any and all subsequent generations formed by cell division. It is understood that not all progeny may be identical due to deliberate or inadvertent mutations. A host cell may be “transfected” or “transformed” which refers to a process by which exogenous nucleic acid is transferred or introduced into the host cell.

The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### III. Preparation of Devices and Extracts

The biological devices described herein are useful in the production of compositions and extracts that can be used to measure glucose levels in a subject. Once the DNA construct has been incorporated into the host cell, the cells are cultured such that the cells multiply. A satisfactory microbiological culture contains available sources of hydrogen donors and acceptors, carbon, nitrogen, sulfur, phosphorus, inorganic salts, and, in certain cases, vitamins or other growth-promoting substances. For example, the addition of peptone provides a readily-available source of nitrogen and carbon. Furthermore, the use of different types of media results in different growth rates and different stationary phase densities; stationary phase is where secondary metabolite production occurs most frequently. A rich media results in a short doubling time and higher cell density at stationary phase. Minimal media results in slow growth and low final cell densities. Efficient agitation and aeration increase final cell densities.

In one aspect, host cells may be cultured or fermented by any method known in the art. The skilled practitioner will be able to select a culture medium based on the species and/or strain of host cell selected. In certain aspects, the culture medium will contain a carbon source. A variety of carbon sources are contemplated, including, but not limited to: monosaccharides such as glucose and fructose, disaccharides such as lactose or sucrose, oligosaccharides, polysaccharides such as starch, or mixtures thereof. Unpurified mixtures extracted from feedstocks are also contemplated as carbon sources, as are one-carbon substrates such as carbon dioxide and/or methanol in the cases of compatible organisms. The carbon source utilized is limited only by the particular organism being cultured.

Culturing or fermenting of host cells may be accomplished by any technique known in the art. In one aspect, batch fermentation may be conducted. In batch fermentation, the composition of the culture medium is set at the beginning and the system is closed to future artificial alterations. In some aspects, a limited form of batch fermentation may be carried out wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial host cells are immobilized on a substrate. Fermentation may be carried out on any scale and may include methods in which literal “fermentation” is carried out as well as other culture methods that are non-fermentative.

In one aspect, the method involves growing the biological devices described herein for a sufficient time to produce SNF3, OGT, and OGlcNA. The ordinary artisan will be able to choose a culture medium and optimum culture conditions based on the biological identity of the host cells.

In certain aspects, after culturing the biological device to produce the proteins of interest, the host cells of the device can be lysed with one or more enzymes to produce an extract. For example, when the host cells are yeast, the yeast cells can be lysed with lyticase. In one aspect, the lyticase concentration can be 500, 600, 700, 800, 900, or 1000 μL per liter of culture, where any value can be the lower or upper endpoint of a range (e.g., 500 to 900 μL, 600 to 800 μL, etc.).

In addition to enzymes, other components can be used to facilitate lysis of the host cells. In one aspect, chitosan can be used in combination with an enzyme to lyse the host cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from 60% to about 100%, 70% to 90%, 75% to 85%, or is about 80% acetylated. The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units. In one aspect, the chitosan can be added until a concentration of 0.0015, 0.0025, 0.0050, 0.0075, 0.01, 0.015, 0.02, 0.03, 0.04, or 0.05% (v/v) is achieved in the culture, where any value can be a lower or an upper end-point of a range (e.g., 0.005 to 0.02%, 0.0075 to 0.015%, etc.). Still further in this aspect, the chitosan is present at a concentration of 0.01%.

In a further aspect, a composition composed of SNF3, OGT, OGlcNA, and optionally a reporter protein can be collected, separated from the microbial cells (lysed or intact), and/or purified through any technique known in the art such as, for example, extraction, precipitation, ultracentrifugation, filtration, size exclusion chromatography, ion exchange chromatography, affinity chromatography, high-pressure liquid chromatography, electrophoresis, any other technique known in the art, or a combination thereof. In an alternative aspect, the microbial cells secrete the proteins of interest into the culture medium.

In one aspect, compositions composed of the proteins of interest with lysed and/or intact host cells can be used herein where it is not necessary to separate the host cells and other components from the proteins.

In one aspect, provided herein are extracts containing SNF3, OGT, OGlcNA, and optionally a reporter protein. In a further aspect, the extracts are collected from cultures or culture media of host cells without lysing the cells. Further in this aspect, the cells can be filtered out or can be incorporated into the extract. In an alternative aspect, the extracts are collected from lysed cells and may be further purified. Exemplary procedures for producing and purifying the extracts are provided in the Examples.

### IV. Applications of the Devices and Extracts

The biological devices and extracts produced therefrom can be used to detect and quantify glucose levels of a subject. As will be discussed below, elevated levels of glucose are associated with several serious diseases and medical conditions.

In one aspect, elevated levels of blood glucose are associated with diabetes. In another aspect, levels of blood glucose below those of a diabetic patient but elevated compared to the normal population average are associated with pre-diabetes and/or metabolic syndrome, both conditions that can develop into diabetes without treatment and/or lifestyle changes.

In one aspect, described herein is a method for measuring glucose levels in a subject, the method involving the steps of:
(a) admixing a sample from the subject with the extract or composition produced from a biological device described herein to produce a test sample;
(b) measuring fluorescence intensity of the test sample; and
(c) correlating the fluorescence produced by the sample to the amount of glucose present in the sample.

In another aspect, described herein is a method for diagnosing or predicting a disease associated with elevated glucose levels in a subject, the method involving the steps of:
(a) admixing a sample from the subject with the extract or composition produced from a biological device described herein to produce a test sample;
(b) measuring fluorescence intensity of the test sample; and
(c) correlating the fluorescence produced by the sample to the disease.

In one aspect, the sample from the subject can be blood, serum, plasma, saliva, urine, or a combination thereof. In another aspect, the sample can be obtained in a non-invasive manner and/or a manner that will not cause pain for a subject. In a further aspect, the sample does not require specialized equipment (e.g., lancet, lancing device, etc.) to obtain. In one aspect, the sample is saliva.

In a further aspect, biological devices, extracts, and compositions such as those described herein are used in diagnostic tests for the detection of diabetes (type 1 or 2) in living patients, including in the early stages of the disease (i.e., pre-diabetes or metabolic syndrome). In this aspect, the use of these diagnostic tests can identify at-risk patients at an early stage, when few or no symptoms are present, thus enabling physicians to start treatment early and delay the progression of the disease.

After the patient sample has been mixed with the extract or composition produced from the biological device, the amount of fluorescence that is subsequently produced is quantified using techniques and instrumentation known in the art. In one aspect, the amount of fluorescence that is produced can be correlated with glycemia (i.e., blood sugar) values from clinical blood sugar tests.

In one aspect, after the fluorescence has been quantified, the value is correlated in order to determine if (1) the subject has diabetes or (2) if the subject has pre-diabetes or is likely to develop diabetes. In one aspect, a chart or computer program can be used to correlate different fluorescence values to different symptoms or probabilities of having or contracting diabetes. Results from a series of such tests on diabetic, pre-diabetic, and healthy patients are presented in the Examples. In one aspect, the amount of fluorescence that is produced can be correlated with glycemia (i.e., blood sugar) values associated with diabetes or the onset of diabetes (i.e., pre-diabetes).

In one aspect, a clinician or medical provider may diagnose a patient with diabetes based on the presence of high fasting blood sugar levels. In a further aspect, the results from use of the devices, compositions, and methods described herein also correlated with results from clinical blood glucose tests. In a still further aspect, early diagnosis of pre-diabetes conditions paves the way for therapeutic, medical, pharmaceutical, and/or lifestyle intervention to delay or prevent the onset of diabetes.

### V. Aspects

The following listing of exemplary aspects supports and is supported by the disclosure provided herein.
Aspect 1: A DNA construct comprising the following genetic components: (a) a gene that expresses SNF3, (b) a gene that expresses an O-linked N-acetylglucosamine transferase (OGT), (c) a gene that expresses an O-linked N-acetylglucosamine-selective-N-acetyl-β-D-glucosaminidase (OGlcNa), and (d) optionally a gene that expresses a fluorescent reporter protein.
Aspect 2: The DNA construct of aspect1, wherein the gene that expresses SNF3 has one of SEQ ID NO. 1 or 6-15 or at least 70% homology thereto.
Aspect 3: The DNA construct of aspect 1, wherein the gene that expresses SNF3 has SEQ ID NO. 1 or at least 70% homology thereto.
Aspect 4: The DNA construct in any one of aspects 1-3, wherein the gene that expresses O-linked N-acetylglucosamine transferase has SEQ ID NO. 2 or at least 70% homology thereto.
Aspect 5: The DNA construct in any one of aspects 1-3, wherein the gene that expresses O-linked N-acetylglucosamine-selective-N-acetyl-β-D-glucosaminidase has SEQ ID NO. 3 or at least 70% homology thereto.
Aspect 6: The DNA construct in any one of aspects 1-5, wherein the construct further comprises a gene that expresses a fluorescent reporter protein.
Aspect 7: The DNA construct aspect 6, wherein the fluorescent reporter protein is a red fluorescent protein, a green fluorescent protein, a cyan fluorescent protein, or a yellow fluorescent protein.
Aspect 8: The DNA construct of aspect 6, wherein the fluorescent reporter protein is enhanced green fluorescent protein.
Aspect 9: The DNA construct aspect 6, wherein the gene that expresses the reporter protein has SEQ ID NO. 4 or 27 or at least 70% homology thereto.
Aspect 10: The DNA construct of aspect 6, wherein the gene that expresses the reporter protein has SEQ ID NO. 4 or at least 70% homology thereto.
Aspect 11: The DNA construct in any one of aspects 1-10, further comprising one or more promoters.
Aspect 12: The DNA construct of aspect 11, wherein the promoter is a T3 promoter, a T7 promoter, an iron promoter, a GAL1 promoter, or a glucose promoter.
Aspect 13: The DNA construct of aspect 11, wherein the promoter is a GAL1 promoter.
Aspect 14: The DNA construct of aspect 11, wherein the promoter is a GAL1 promoter, wherein the GAL1 promoter precedes each genetic component.
Aspect 15: The DNA construct of aspect 11, wherein the promoter has one of SEQ ID NO. 16-19 or at least 70% homology thereto.
Aspect 16: The DNA construct in any one of aspects 1-15, further comprising at least one terminator.
Aspect 17: The DNA construct of aspect 16, wherein the terminator is a T7 terminator or a CYC1 terminator.
Aspect 18: The DNA construct of aspect 16, wherein the terminator is a CYC1 terminator, wherein the CYC1 terminator follows each genetic component.
Aspect 19: The DNA construct of aspect 16, wherein the terminator has SEQ ID NO. 20 or at least 70% homology thereto.
Aspect 20: The DNA construct in any one of aspects 1-19, further comprising a gene that expresses a riboswitch.
Aspect 21: The DNA construct of aspect 20, wherein the riboswitch has one of SEQ ID NO. 21-23 or at least 70% homology thereto.
Aspect 22: The DNA construct in any one of aspects 1-21, further comprising a gene that expresses a ribosomal binding site.
Aspect 23: The DNA construct of aspect 22, wherein the ribosomal binding site has SEQ ID NO. 24-26 or at least 70% homology thereto.
Aspect 24: The DNA construct in any one of aspects 1-23, wherein the DNA construct comprises from 5′ to 3′ the following genetic components in the following order: (a) a gene that expresses SNF3, (b) a gene that expresses O-linked N-acetylglucosamine transferase, and (c) a gene that expresses O-linked N-acetylglucosamine-selective-N-acetyl-β-D-glucosaminidase.
Aspect 25: The DNA construct in any one of aspects 1-23, wherein the DNA construct comprises from 5′ to 3′ the following genetic components in the following order: (a) a gene that expresses SNF3, (b) a gene that expresses O-linked N-acetylglucosamine transferase, (c) a gene that expresses O-linked N-acetylglucosamine-selective-N-acetyl-β-D-glucosaminidase, and (d) a gene that expresses a reporter protein.
Aspect 26: The DNA construct in any one of aspects 1-23, wherein the DNA construct comprises from 5′ to 3′ the following genetic components in the following order: (a) a gene that expresses SNF3, (b) a CYC1 terminator, (c) a GAL1 promoter, (d) a gene that expresses O-linked N-acetylglucosamine transferase, (e) a CYC1 terminator, (f) a GAL1 promoter, (g) a gene that expresses O-linked N-acetylglucosamine-selective-N-acetyl-β-D-glucosaminidase, (h) a CYC1 terminator, (i) a GAL1 promoter, and (j) a gene that expresses a reporter protein.
Aspect 27: The DNA construct in any one of aspects 1-23, wherein the DNA construct comprises from 5′ to 3′ the following genetic components in the following order: (a) a gene that expresses SNF3 having SEQ ID NO. 1 or at least 70% homology thereto, (b) a CYC1 terminator, (c) a GAL1 promoter, (d) a gene that expresses O-linked N-acetylglucosamine transferase having SEQ ID NO. 2 or at least 70% homology thereto, (e) a CYC1 terminator, (f) a GAL1 promoter, (g) a gene that expresses O-linked N-acetylglucosamine-selective-N-acetyl-β-D-glucosaminidase having SEQ ID NO. 3 or at least 70% homology thereto, (h) a CYC1 terminator, (i) a GAL1 promoter, and (j) a gene that expresses a reporter protein having SEQ ID NO. 4 or at least 70% homology thereto.
Aspect 28: The DNA construct of aspect 1, wherein the DNA construct has SEQ ID NO. 5 or at least 70% homology thereto.
Aspect 29: The DNA construct in any one of aspects 1-28, wherein the DNA construct further comprises a gene that confers resistance to an antibiotic.
Aspect 30: The DNA construct of aspect 29, wherein the antibiotic comprises tetracycline, neomycin, kanamycin, ampicillin, hygromycin, chloramphenicol, amphotericin B, bacitracin, carbapenam, cephalosporin, ethambutol, fluoroquinolones, isoniazid, methicillin, oxacillin, vancomycin, streptomycin, quinolones, rifampin, rifampicin, sulfonamides, cephalothin, erythromycin, gentamycin, penicillin, other commonly-used antibiotics, or a combination thereof.
Aspect 31: A vector comprising the DNA construct in any one of aspect 1-30.
Aspect 32: The vector of aspect 31, wherein the vector is a plasmid.
Aspect 33: The vector of aspect 32, wherein the plasmid is pWLneo, pSV2cat, pOG44, pXT1, pSG, pSVK3, pBSK, pBSKII, pYES, pYES2, pUC, pUC19, or pETDuet-1.
Aspect 34: The vector of aspect 32, wherein the plasmid is pYES2.
Aspect 35: A biological device comprising host cells transformed with the DNA construct in any one of aspects 1-30 or the vector in any one of aspects 31-34.
Aspect 36: The biological device of aspect 35, wherein the host cells comprise fungi or bacteria.
Aspect 37: The biological device of aspect 36, wherein the bacteria comprise E. coli.
Aspect 38: The biological device of aspect 36, wherein the fungi comprise yeast.
Aspect 39: The biological device of aspect 38, wherein the yeast comprise S. cerevisiae.
Aspect 40: An extract produced by culturing the biological device of aspects 35-39.
Aspect 41: The extract of aspect 40, wherein the extract is produced by:
   (a) culturing the biological device in a culture medium; and
   (b) removing the extract from the culture medium.
Aspect 42: A composition comprising SNF3, O-linked N-acetylglucosamine transferase, O-linked N-acetylglucosamine-selective-N-acetyl-β-D-glucosaminidase, and optionally a fluorescent reporter protein.
Aspect 43: A method for quantifying the amount of glucose in a subject, the method comprising:
   (a) admixing the extract of aspects 40-41 or the composition of aspect 42 with a sample from the subject to produce a test sample;
   (b) measuring the fluorescence produced by the test sample; and
   (c) correlating the fluorescence produced by the test sample to the amount of glucose present in the sample.
Aspect 44: The method of aspect 43, wherein the fluorescence produced by the test sample is correlated to the amount of glucose present in the blood of the subject.
Aspect 45: A method for diagnosing or predicting a disease associated with elevated levels of glucose in a subject, the method comprising the steps of:
   (a) admixing the extract of aspects 40-41 or the composition of aspect 42 with a sample from the subject to produce a test sample;
   (b) measuring the fluorescence produced by the test sample; and
   (c) correlating the fluorescence produced by the test sample to the disease.
Aspect 46: The method of aspect 45, wherein the fluorescence produced by the test sample is correlated to the amount of glucose present in the blood of the subject that is associated with the disease.
Aspect 47: The method in any one of aspects 43-46, wherein the sample comprises blood, serum, plasma, saliva, urine, or a combination thereof.
Aspect 48: The method in any one of aspects 43-46, wherein the sample is saliva.
Aspect 49: The method in any one of aspects 45-48, wherein the disease is diabetes, pre-diabetes, or metabolic syndrome.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperatures, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions) can be used to optimize the product purity and yield obtained from the desired process. Only reasonable and routine experimentation will be required to optimize such processes and conditions.

### I. Preparation of DNA Construct

The genes described below were assembled in pYES2 plasmid vectors and included an array of promoters and terminators. The full sequence of the DNA-based glucose sensor comprises an arrangement of genes, Gall promoters, and CYC1 terminators in the following order: SNF3+CYC1 Terminator+GAL1 Promoter+OGT+CYC1 Terminator+GAL1 Promoter+OGlcNA+CYC1 Terminator+GAL1 Promoter+EGFP as provided in FIGS. 1 and 2.

Sequences included the SNF3 glucose receptor with Genbank accession number XM_001386379.1 (a high affinity glucose receptor already proven as a glucose sensor), O-linked acetylglucosamine transferase (OGT) with Genbank accession number EAX05286.1, O-GlcNAc selective N-Acetyl-beta-D-glucosaminidase (O-GlcNAc) having Genbank accession number NP_571979.1, and Enhanced Green Fluorescent Protein (EGFP) having Genbank accession number ACV20892.1.

Each gene was PCR amplified using gene-specific overlap primers and assembled sequences were subcloned into a HingIII-and XbaI-digested pYES2 vector. PCR amplified pieces of all fragments were combined by using homologous recombination technology (Gibson Assembly). Clones obtained after transformation were sequenced and analyzed for DNA accuracy. At the completion of PCR of all four targeted genes, homologous recombination of the glucose detection device, and sub-cloning into pYES2 vector, four clones were selected from a transformed plate and processed for full length DNA sequencing. A clone with 100% matching sequence corresponding to the construct design was selected and purified to obtain plasmid construct at a mid-scale purification level. This DNA device will allow the detection of proteins related to the presence of glucose in patient saliva samples as well as to detect existence of glucose itself.

### II. Host Cell Purification and Transformation

The biological device was constructed using yeast (S. cerevisiae) cells with a pYES2 plasmid vector. After the vector comprising the DNA construct has been produced, the resulting vector can be incorporated into the host cells using methods known in the art (e.g., Gietz, R. D. and R. H. Schiestl, 2007, Nature Protocols, “Quick and easy yeast transformation using the LiAc/SS carrier and DNA/PEG method,” Vol. 2, 35-37). INVSc1 competent yeast cells (Invitrogen, Inc.) were used for some transformations. A kit for preparing and transforming INVSc1 cells was purchased from Sigma-Aldrich, Inc., and used according to a protocol provided by the manufacturer. In brief, competent yeast cells were transformed with the DNA construct described herein and selected on synthetic complete (SC) dropout plates (deficient in uracil base). A well isolated clone was picked from the SC plate and preserved in YPD medium containing 15% glycerol for storage at −80° C.

DNA expression and effectiveness of transformation were determined by fluorescence of the transformed cells expressed in fluorescence units (FSUs), according to a protocol provided by the manufacturer, using a 20/20 Luminometer (Promega). The blue fluorescence module (with a 450 nm excitation wavelength and a 600 nm emission wavelength) was used to evaluate the effectiveness of transformation. When no fluorescent reporter protein was assembled, no fluorescence was observed. Plasmid DNA extraction purification, PCR, and gel electrophoresis were also used to confirm transformation.

III. Production of Extracts from the Yeast Device

Transformed S. cerevisiae cells were grown in yeast malt broth at 30° C. for 24 hours with an optimum OD of 2.56. An extract was produced from the yeast culture as follows: fermentation was conducted in yeast malt broth mixed with raffinose and galactose for 30° C. for 72 hours. The cultures were then centrifuged at 9,000 rpm for 12 min at 15° C. Pellets were resuspended in 50 mL of sterile deionized water per gram of pellet. The resulting solutions were sonicated 4 times for a total of 2 minutes and 30 seconds and were then again centrifuged at 9,000 rpm for 12 min at 15° C. Supernatants were decanted and filtered with 0.56 μm filters and stored for later use.

### IV. Sample Collection and Processing

Saliva samples from fasting patients were collected by having the patients spit in a 3 cm×2 cm collection tube until a total saliva volume of 3 mL per patient was collected. These samples were mixed with the DNA-based glucose sensors prepared as described above, at different ratios, and subjected to vortexing for 30-50 seconds. Although different ratios of the yeast DNA-based glucose sensors to saliva were used (i.e., 1:1, 2:1, 3:1), the ratio 3:1 showed the best efficacy for each sample (i.e., 190 μL of extract: 30 μL of saliva).

A total volume of 200 μL of saliva alone, DNA-based glucose sensor (DGS) alone, or DGS+saliva was used for fluorescence detection. The detection was performed using a Glomax E-8032: Multi+Detection System with Instinct™ Software: Base Instrument with Shaking (PROMEGA, Madison, Wisconsin USA) by placing the selected sample on a plate reader, and the samples were exposed to a light source using the blue module (Ex: 490 nm, Em: 510-570 nm). Other excitation wavelengths were tested; however the selected blue light module showed the best consistent results. Fluorescence was determined based on fluorescence units (FSU), and compared to the results of the conventional glycemia tests. The natural inherent fluorescence (1,900 FSU) of the yeast was subtracted from the final fluorescence of each sample.

### V. Clinical Analysis of Gylcemia

Glycemia levels for both diabetic and non-diabetic subjects were determined quantitatively following standard in vitro diagnostic protocols using a colorimetric technique in Mindray BS380 equipment. Patients were classified as diabetic, pre-diabetic, or healthy based on guidelines established by the American Diabetes Association and the World Health Organization. Blood glucose ranges (in mg/dL) for each group are provided in Table 4.

TABLE 4
Criteria for Diagnosing Diabetes and Related Conditions

| Condition | Glucose Level (mg/dL) |
|---|---|
| Diabetic | >126 |
| Pre-Diabetic | 100-125 |
| Healthy | <100 |

### VI. Statistical Analysis

The statistical data was analyzed using Microsoft Excel 2013. An analysis of variance (ANOVA) was performed to determine the equal value of the means in the groups of patients and a linear regression was carried out in order to relate the study variables, in this case glycemia and fluorescence (extract+saliva of patients).

VII. Correlation of Glycemia with Fluorescence

Saliva samples mixed with the extracts produced from the biological device from patients with high clinical glycemia (i.e., diagnosed diabetics) exhibited higher fluorescence intensity than patients with low clinical glycemia after background correction for fluorescence intensity of the extracts themselves (4600 FSU). Based on comparative fluorescence intensity and clinical results, patients were classified into three groups. Results are presented in Table 5.

TABLE 5
Results of fluorescence intensity and glycemia of patients
placed in the following groups: Group 1 = Diagnosed
Diabetics, Group 2 = Pre-Diabetics, and Group 3 = Healthy

| | | | | |
|---|---|---|---|---|
| | | DNAGYE + | DNAGYE + | |
| | | Saliva Initial | Saliva Final | Clinical |
| Patient | | Fluorescence | Fluorescence | Glycemia |
| Age | Gender | (FSU) | (FSU) | (mg/dL) |

Group 1: Diabetics Mean = 6957.143 +/− 822.3

| | | | | |
|---|---|---|---|---|
| 36 | Female | 10600 | 6000 | 127 |
| 38 | Female | 11000 | 6400 | 135 |
| 39 | Female | 11200 | 6600 | 129 |
| 31 | Female | 11300 | 6700 | 132 |
| 30 | Male | 11500 | 6900 | 172 |
| 35 | Female | 12300 | 7700 | 152 |
| 44 | Female | 13000 | 8400 | 161 |

Group 2: Pre-Diabetics. Mean = 5500 +/− 308.22

| | | | | |
|---|---|---|---|---|
| 23 | Male | 9700 | 5100 | 117 |
| 42 | Male | 10000 | 5400 | 102 |
| 40 | Female | 10000 | 5400 | 123 |
| 37 | Male | 10300 | 5700 | 125 |
| 45 | Male | 10500 | 5900 | 123 |

Group 3: Healthy Mean = 3600 +/− 930.6

| | | | | |
|---|---|---|---|---|
| 4 | Female | 7000 | 2400 | 65 |
| 18 | Female | 7300 | 2700 | 77 |
| 1 | Female | 7500 | 2900 | 84 |
| 12 | Female | 7500 | 2900 | 84 |
| 3 | Female | 7600 | 3000 | 73 |
| 14 | Female | 7700 | 3100 | 77 |
| 28 | Female | 8600 | 4000 | 84 |
| 24 | Female | 9000 | 4400 | 89 |
| 43 | Female | 9000 | 4400 | 93 |
| 6 | Female | 9500 | 4900 | 97 |
| 25 | Female | 9500 | 4900 | 97 |

Note:
DNAGYE: DNA Glucose Yeast Extract, FSU: Fluorescence units
* “DNAGYE + Saliva Final Fluorescence (FSU)” is the fluorescence background corrected for extract fluorescence (4600 FSU)
Note 2:
Results are based on the mean of number of patients per group, after ANOVA.
Note 3:
p-value = 1.5 × 10⁻⁷

Fluorescence reading ranges were established based on the laboratory and clinical findings; these are presented in Table 6.

TABLE 6
Ranges of fluorescence intensity and glycemia levels
according the patient groups 1, 2, and 3.

| | | Range of Fluorescence |
|---|---|---|
| Group | Range of glycemia | (FSU) |
| Diabetic | >126 mg/dL | 10,600-13,000 |
| Pre-Diabetic | 100 mg/dL-125 mg/dL | 9,700-10.500 |
| Healthy | <100 mg/dL | 6,000-9,500 |

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A method for quantifying the amount of glucose in a subject, the method comprising:
(a) admixing an extract produced by a biological with a sample from the subject to produce a test sample, wherein biological device comprising host cells transformed with a DNA construct, wherein the DNA construct comprises the following genetic components: (a) a gene that expresses SNF3, (b) a gene that expresses an O-linked N-acetylglucosamine transferase (OGT), (c) a gene that expresses an O-linked N-acetylglucosamine-selective-N-acetyl-β-D-glucosaminidase (OGlcNa), and (d) optionally a gene that expresses a fluorescent reporter protein;
(b) measuring the fluorescence produced by the test sample; and
(c) correlating the fluorescence produced by the test sample to the amount of glucose present in the sample.

2. The method of claim 1, wherein the DNA construct comprises from 5′ to 3′ the following genetic components in the following order: (a) a gene that expresses SNF3, (b) a gene that expresses O-linked N-acetylglucosamine transferase, and (c) a gene that expresses O-linked N-acetylglucosamine-selective-N-acetyl-β-D-glucosaminidase.

3. The method of claim 1, wherein the DNA construct comprises from 5′ to 3′ the following genetic components in the following order: (a) a gene that expresses SNF3, (b) a gene that expresses O-linked N-acetylglucosamine transferase, (c) a gene that expresses O-linked N-acetylglucosamine-selective-N-acetyl-β-D-glucosaminidase, and (d) a gene that expresses a reporter protein.

4. The method of claim 1, wherein the gene that expresses SNF3 has one of SEQ ID NO. 1 or 6-15 or at least 90% identity thereto.

5. The method of claim 1, wherein the gene that expresses O-linked N-acetylglucosamine transferase has SEQ ID NO. 2 or at least 90% identity thereto.

6. The method of claim 1, wherein the gene that expresses O-linked N-acetylglucosamine-selective-N-acetyl-β-D-glucosaminidase has SEQ ID NO. 3 or at least 90% identity thereto.

7. The method of claim 1, wherein the fluorescence produced by the test sample is correlated to the amount of glucose present in the blood of the subject.

8. The method of claim 1, wherein the sample comprises blood, serum, plasma, saliva, urine, or a combination thereof.

9. The method of claim 1, wherein the sample is saliva.

10. A method for diagnosing or predicting a disease associated with elevated levels of glucose in a subject, the method comprising the steps of:
(a) admixing an extract produced by a biological with a sample from the subject to produce a test sample, wherein biological device comprising host cells transformed with a DNA construct, wherein the DNA construct comprises the following genetic components: (a) a gene that expresses SNF3, (b) a gene that expresses an O-linked N-acetylglucosamine transferase (OGT), (c) a gene that expresses an O-linked N-acetylglucosamine-selective-N-acetyl-β-D-glucosaminidase (OGlcNa), and (d) optionally a gene that expresses a fluorescent reporter protein;
(b) measuring the fluorescence produced by the test sample; and
(c) correlating the fluorescence produced by the test sample to the disease.

11. The method of claim 10, wherein the DNA construct comprises from 5′ to 3′ the following genetic components in the following order: (a) a gene that expresses SNF3, (b) a gene that expresses O-linked N-acetylglucosamine transferase, and (c) a gene that expresses O-linked N-acetylglucosamine-selective-N-acetyl-β-D-glucosaminidase.

12. The method of claim 10, wherein the DNA construct comprises from 5′ to 3′ the following genetic components in the following order: (a) a gene that expresses SNF3, (b) a gene that expresses O-linked N-acetylglucosamine transferase, (c) a gene that expresses O-linked N-acetylglucosamine-selective-N-acetyl-β-D-glucosaminidase, and (d) a gene that expresses a reporter protein.

13. The method of claim 10, wherein the gene that expresses SNF3 has one of SEQ ID NO. 1 or 6-15 or at least 90% identity thereto.

14. The method of claim 10, wherein the gene that expresses O-linked N-acetylglucosamine transferase has SEQ ID NO. 2 or at least 90% identity thereto

15. The method of claim 10, wherein the gene that expresses O-linked N-acetylglucosamine-selective-N-acetyl-β-D-glucosaminidase has SEQ ID NO. 3 or at least 90% identity thereto.

16. The method of claim 10, wherein the fluorescence produced by the test sample is correlated to the amount of glucose present in the blood of the subject that is associated with the disease.

17. The method of claim 10, wherein the sample comprises blood, serum, plasma, saliva, urine, or a combination thereof.

18. The method of claim 10, wherein the sample is saliva.

19. The method of claim 10, wherein the disease is diabetes, pre-diabetes, or metabolic syndrome.

20. A glucose sensor comprising an extract produced by a biological with a sample from the subject to produce a test sample, wherein biological device comprising host cells transformed with a DNA construct, wherein the DNA construct comprises the following genetic components: (a) a gene that expresses SNF3, (b) a gene that expresses an O-linked N-acetylglucosamine transferase (OGT), (c) a gene that expresses an O-linked N-acetylglucosamine-selective-N-acetyl-β-D-glucosaminidase (OGlcNa), and (d) optionally a gene that expresses a fluorescent reporter protein.
